(19) United States
(12) Patent Application Publication
(10) Pub. No.: US 2024/0091391 A1
(43) Pub. Date: Mar. 21, 2024

(54) MOF FOR RADIOTHERAPY

(71) Applicant: Node Pharma AS, Oslo (NO)

(72) Inventors: Sigurd ØIEN-ØDEGAARD, Oslo (NO); Erik MJÅLAND, Oslo (NO)

(73) Assignee: Node Pharma AS, Oslo (NO)

(21) Appl. No.: 18/384,509

(22) Filed: Oct. 27, 2023

Related U.S. Application Data
(63) Continuation of application No. PCT/NO2022/050169, filed on Jul. 7, 2022.

(30) Foreign Application Priority Data
Jul. 9, 2021 (NO) 20210895

(51) Int. Cl. A61K 51/12; A61K 51/10

(52) U.S. Cl. CPC A61K 51/1244; A61K 51/1027; A61K 51/1045; A61K 51/1051

(57) ABSTRACT

The present invention relates to MOFs for use in radiotherapy. More particularly, the invention provides a particle comprising a MOF, optionally at least one targeting moiety and at least one radionuclide; a composition comprising said particle; said particle or composition for use as a medicament; said particle or composition for use in a method for the treatment of a proliferative disease; said particle or composition for use in a method for the treatment of a chronic inflammatory disease; and a kit comprising a particle comprising a MOF, an optional targeting moiety, and a radionuclide cation.

### FIELD OF THE INVENTION

The present invention relates to MOFs for use in radiotherapy. More particularly, the invention provides a particle comprising a MOF and at least one radionuclide; a particle comprising a MOF, at least one targeting moiety and at least one radionuclide; a composition comprising said particle; said particle or composition for use as a medicament; said particle or composition for use in a method for the treatment of a proliferative disease; said particle or composition for use in a method for the treatment of a chronic inflammatory disease; and a kit comprising a particle comprising a MOF and optionally a targeting moiety, and a radionuclide cation.

### BACKGROUND OF THE INVENTION

Cancer is a widespread group of diseases that affect millions of people, either directly or indirectly. WHO estimated 19.3 million new cases of cancer in 2020, and 50.6 million individuals living with a 5-year prevalence. Despite decades of improvement in treatments, the American Cancer Society expects the number of new incidents of cancer to grow to 27.5 million per year by 2040, and the number of annual deaths from cancer to increase to 16.2 million. Common methods of cancer treatment include surgery, chemotherapy and external beam irradiation. However, there is a significant unmet medical need for new and more effective cancer treatment options.

One treatment option, with the potential of high specificity and efficiency, is radioimmunotherapy (RIT). RIT drugs are based on tissue targeting moieties linked to a carrier that can bind and hold on to radioisotopes. The tissue targeting moieties, which frequently are antibodies, are used to bring the radioisotopes into close proximity of the unwanted cell types, such as cancer cells, resulting in local delivery of high energy and highly cytotoxic radiation—killing the unwanted cells and not the healthy surrounding tissue. This type of specific cell killing can be essential not only for the treatment of cancerous diseases such as sarcomas and carcinomas, but also hyperplastic and neoplastic diseases and chronic inflammatory diseases. Further, systemic treatment using RIT can be extremely effective for patients with metastatic tumours, otherwise difficult to reach without detrimental side effects.

Successful delivery of the radiation to the unwanted cells depends on a stable radioisotope-carrier interaction to prevent unanticipated side effects due to radioisotope leakage. However, the carriers currently in use—frequently small-molecule ligands or chelators—do not fulfil these requirements for a range of potent radioisotopes, in particular the alpha-emitting radium isotopes including radium-223 and radium-224. As a consequence, some of the most potent radioisotopes cannot be used in RIT.

A further challenge is that the recoil energy from the emission of radiation, particularly from alpha-emission, in many cases may cause the release of daughter nuclides from the carrier. Such release may occur even if the carrier is capable of complexing the daughter nuclides. Consequently, radioactive daughter nuclides may diffuse away from the carrier, potentially causing systemic toxicity.

Hence, there is a need for improved drugs for radiotherapy.

### BRIEF DESCRIPTION OF THE DRAWINGS

FIG. 1 shows a schematic illustration of an embodiment of the invention.

FIG. 2 shows a schematic representation of a network structure with fcu topology,

FIG. 3 illustrates the pores in fcu MOFs.

FIG. 4 shows an example of a coordinating site in a tetrahedral cage corner.

FIG. 5 shows an example of a coordinating site in an octahedral pore corner.

FIG. 6 shows an example of a coordinating site in an octahedral pore corner.

FIG. 7 shows an example of a coordinating site in an octahedral cage corner.

FIG. 8 shows an example of a pore where several free functional groups are extending into the pore.

FIG. 9 illustrates the MOF UiO-66.

FIG. 10 illustrates the MOF MIL-53.

FIG. 11 illustrates a MOF having a zeolitic imidazolate framework and a so-called sod topology.

FIG. 12 shows a BCA assay of antibody (IgG) conjugated UIO-66(COOH)₂.

FIG. 13 shows that anti-CD37-conjugated MOFs (NP1) bind CD37 positive cells (Duadi cells).

FIG. 14 shows that anti-EpCAM or anti-HER-conjugated UiO-66-(COOH)₂ bind colorectal cancer cells (HTC116 and HT29) expressing both antigens.

FIG. 15 shows anti-HER2-conjugated UiO-66-(COOH)₂ binds HER2 positive cancer cells (JIMT1 cells).

FIG. 16 shows radium 223 (Ra223) adsorption and retention ability of UiO-66-(COOH)₂ in human serum, wherein graph A) provides the Ra223 loading (adsorption), and the graph B) provides the retention.

FIG. 17 shows biodistribution of radium 223 (Ra223) loaded UiO-66-(COOH)₂ in healthy mice.

FIGS. 18A-F show the weight of mice injected with radium 223 (Ra223) loaded UiO-66-(COOH)₂ resulting in side effects with doses of 937 kBq per kg and higher.

FIGS. 19A and B show in vitro cell viability after exposing them to UiO-66-(COOH)₂ for 24 and 48 hours.

### BRIEF SUMMARY OF THE INVENTION

The inventors have discovered that particular metal-organic-framework (MOF) structures comprising certain free functional groups can adsorb and act as stable carriers for selected radionuclides in clinically relevant environment. Furthermore, these MOFs, in the form of particles, can be linked to targeting moieties, enabling targeted delivery of the particles, and thus radionuclides, in vivo. These particles, comprising radionuclides, or comprising both targeting moieties and radionuclides, thus represent promising drug candidates.

In one aspect, the present invention relates to a particle, said particle comprising
a MOF, wherein the MOF comprises
   a repeating three-dimensional network of inorganic monomers and organic monomers, forming pores, and
   at least one free functional group that extends into a pore, wherein the functional group is selected from the group comprising carboxylic acid, carboxylate, hydroxyl, sulfonic acid, sulfonate, sulfhydryl, primary amines, secondary amines, and combinations thereof;
at least one radionuclide, wherein the at least one radionuclide is selected from the group comprising radium-223, radium-224, radium-225, bismuth-212, bismuth-213, lead-212, actinium-225, thorium-227, terbium-149 and terbium-161;

and wherein the at least one radionuclide is located in at least one of the pores.

The present invention also relates to a targeting particle, said particle comprising
a MOF, wherein the MOF comprises
   a repeating three-dimensional network of inorganic monomers and organic monomers, forming pores, and
   at least one free functional group that extends into a pore, wherein the functional group is selected from the group comprising carboxylic acid, carboxylate, hydroxyl, sulfonic acid, sulfonate, sulfhydryl, primary amines, secondary amines, and combinations thereof;
at least one targeting moiety connected to the particle on an external surface of the particle; and
at least one radionuclide, wherein the at least one radionuclide is selected from the group comprising radium-223, radium-224, radium-225, bismuth-212, bismuth-213, lead-212, actinium-225, thorium-227, terbium-149 and terbium-161:

and wherein the at least one radionuclide is located in at least one of the pores.

Accordingly, the particle of the invention optionally comprises at least one targeting moiety connected to the particle on an external surface of the particle.

In a second aspect, the invention relates to a kit, wherein the kit comprises

in a first container,

a particle comprising
a MOF, wherein the MOF comprises
   a repeating three-dimensional network of inorganic monomers and organic monomers, forming pores, and
   at least one free functional group that extends into a pore, wherein the functional group is selected from the group comprising carboxylic acid, carboxylate, hydroxyl, sulfonic acid, sulfonate, sulfhydryl, primary amines, secondary amines, and combinations thereof;

and in a second container,

a radionuclide, wherein the radionuclide is selected from the group comprising radium-223, radium-224, radium-225, bismuth-212, bismuth-213, lead-212, actinium-225, thorium-227, terbium-149 and terbium-161, or a radionuclide generating any one of radium-223, radium-224, radium-225, bismuth-212, bismuth-213, lead-212, actinium-225, thorium-227, terbium-149 and terbium-161 as a daughter.

In one embodiment, the particle further comprises at least one targeting moiety connected to the particle on an external surface of the particle.

In a third aspect, the invention relates to a composition as further disclosed, said composition comprising at least one particle as defined above with at least one pharmaceutically acceptable carrier, diluent, and/or excipient.

In a fourth aspect, the invention relates to said particle or said composition for use as a medicament, as further disclosed herein.

In a fifth aspect, the invention relates to said particle or said composition for use in a method for the treatment of a chronic inflammatory disease, as further disclosed herein.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

Metal-organic frameworks (MOF), also referred to as porous coordination polymers (PCP), are solid compounds, usually in the form of powders, that belong to the substance class of coordination polymers. Chemically, MOFs comprise repeating networks of polytopic inorganic monomers in the form of metal ions or clusters, and organic monomers, also referred to as bridging ligands or linkers. The inorganic and organic monomers are bound by coordinate covalent bonds or ionic bonds between a Lewis acidic metal cation and a Lewis basic organic functional group (e.g. carboxylate, amine, imine). The structures of MOFs are well-tuneable by selection of the organic and inorganic monomers. The organic monomers of a MOF may all be identical, or the MOF may comprise two or more different organic monomers with near-identical geometry of the framework-coordinating functional groups. The latter is referred to as a “mixed-linker MOF”. Analogously, the inorganic monomers of a MOF may also be identical, or they may differ, resulting in a “mixed-metal MOF”.

MOFs may be crystalline, amorphous, or have a deformable structure. MOFs that are made of structurally rigid monomers tend to form repeating three-dimensional networks that feature permanent porosity, i.e. they are crystalline. Such crystalline MOFs feature a number of pores. As used herein, the term “pore” refers to any type of aperture, cavity, channel, hole in the MOF network. A MOF may comprise pores of different shapes in different ratios, based on the crystal structure of the MOF. However, the pores of a particular MOF are generally quite similar to each other in respect to size and chemical environment.

The organic monomer of a MOF may contain functional groups that are not interacting directly with the inorganic monomers to form the MOF network. In the present disclosure, such functional groups are referred to as “free” functional groups. If such functional groups are present in a crystalline MOF, these may be directed into the pores of the MOF, creating a site for adsorption, coordination and/or chelation of a guest ion or molecule. The precise nature of such a site depends on the network topology (geometry) of the MOF and the type and number of functional groups directed into the pore. By selecting the appropriate MOF in terms of e.g. free functional groups, specific chelating sites, such as in the pores, can be constructed for strong, selective adsorption, coordination and/or chelation, of particular compounds. As used herein, the terms “chelating” and “chelation” refer to the coordination to, complexing of and/or binding of a metal, such as a radionuclide cation, by at least two ligands. The term “ligand” refers to a moiety, such as a molecule, a part of a molecule, a functional group, capable of coordinating to, complexing and/or binding a metal.

Different functional groups can be used to modify the geometry and charge balance of the chelating site. Since MOFs are built from coordination bonds between the organic and inorganic monomers, additional functional groups which may act as coordinating groups can produce undesired and/or unexpected products in the preparation of the MOF. Therefore, the introduction of free functional groups in a MOF requires careful design.

In the present invention, particles of specific types of MOFs are used as carriers for particular radionuclides. The MOFs used in the present invention are crystalline MOFs comprising potentially metal-coordinating free functional groups extending into pores of the MOF in such a manner that said free functional groups can coordinate to and/or bind a radionuclide cation, such as a radionuclide cation. Hence, each pore of these MOFs has the ability of functioning as a chelating macro ligand for a radionuclide. The MOF particles represent a new class of carriers, and may have the ability to contain potent radioisotopes, with little or no leakage, thus improving the potential of RIT and providing a new option for the treatment of proliferative diseases such as cancer.

Thus, in one aspect, the invention relates to a particle comprising a MOF, wherein the MOF comprises a repeating three-dimensional network of inorganic monomers and organic monomers, forming pores, wherein the pores are designed for chelating a radionuclide, and wherein the particle comprises a radionuclide located in at least one of the pores. Optionally, the particle comprises at least one targeting moiety. Hence, in one embodiment, the particle comprises at least one targeting moiety connected to the particle on an external surface of the particle.

In some embodiments, the invention relates to a particle comprising
a MOF, wherein the MOF comprises
   a repeating three-dimensional network of inorganic monomers and organic monomers, forming pores, and
   at least one free functional group that extends into a pore, wherein the functional group is selected from the group comprising carboxylic acid, carboxylate, hydroxyl, sulfonic acid, sulfonate, sulfhydryl, primary amines, secondary amines, and combinations thereof; and
optionally at least one targeting moiety connected to the particle on an external surface of the particle; and
at least one radionuclide, wherein the at least one radionuclide is selected from the group comprising radium-223, radium-224, radium-225, bismuth-212, bismuth-213, lead-212, actinium-225, thorium-227, terbium-149 and terbium-161:

and wherein the at least one radionuclide is located in at least one of the pores.

In some embodiments, the invention relates to a particle comprising
a MOF, wherein the MOF comprises
   a repeating three-dimensional network of inorganic monomers and organic monomers, forming pores, and
   at least one free functional group that extends into a pore, wherein the functional group is selected from the group comprising carboxylic acid, carboxylate, hydroxyl, sulfonic acid, sulfonate, sulfhydryl, primary amines, secondary amines, and combinations thereof; and
optionally at least one targeting moiety connected to the particle on an external surface of the particle; and
at least one radionuclide cation, wherein the at least one radionuclide is selected from the group comprising radium-223, radium-224, radium-225, bismuth-212, bismuth-213, lead-212, and thorium-227:

and wherein the at least one radionuclide cation is located in at least one of the pores.

In some embodiments, the invention relates to a particle comprising
a MOF, wherein the MOF comprises
   a repeating three-dimensional network of inorganic monomers and organic monomers, forming pores, and
   at least one free functional group that extends into a pore, wherein the functional group is selected from the group comprising carboxylic acid, carboxylate, hydroxyl, sulfonic acid, sulfonate, sulfhydryl, and combinations thereof; and
optionally at least one targeting moiety connected to the particle on an external surface of the particle; and
at least one radionuclide cation, wherein the at least one radionuclide is selected from the group comprising radium-223, radium-224, radium-225, bismuth-212, bismuth-213, lead-212, actinium-225, and thorium-227:

and wherein the at least one radionuclide cation is located in at least one of the pores.

In these embodiments, at least one targeting moiety may be connected to the particle on an external surface of the particle.

FIG. 1 illustrates schematically a non-limiting embodiment of a particle 1 of the invention, comprising a MOF 2 forming pores 3, and having functional groups 4 extending into pores 3, wherein the particle 1 comprises targeting moieties 5, here shown as antibodies, and wherein a radionuclide 6 is located in a pore 3.

The MOFs of the invention comprise a repeating three-dimensional network of inorganic monomers and organic monomers, i.e. they are crystalline. The organisation of the inorganic monomers and organic monomers in a repeating three-dimensional network leads to the formation of pores; i.e. the rigidity and set geometry of the inorganic and organic monomers leads to a set spatial separation between certain monomers such that pores are formed. MOFs with free functional groups can be seen as having a somewhat dynamic pore size due to the freedom of orientation of the free functional groups. Pore size may e.g. be measured by “inclusion sphere”, i.e. the largest sphere that can be placed in a pore without the sphere contacting the van der Waals surface of the MOF. The size of pore openings, also referred to as “window size”, may be measured by “diffusion sphere”, i.e. the smallest sphere that can move through the structure. A MOF for use in the invention advantageously has a pore size of 0.3-3 nm inclusion sphere, preferably 0.5-1.5 nm inclusion sphere. Further, the MOFs of the invention have a pore window of a certain size to allow the adsorption of radionuclides, preferably a diffusion sphere with diameter of at least 3 Å. The pore size may be controlled by the size, structure and bonding of the inorganic monomers and the organic monomers. The pores may be of any shape, including, but not limited to, tetrahedral, octahedral, hexagonal.

MOFs based on Zr(IV), Hf(IV), Fe(III), Al(III), Ti(IV), Cr(III) with carboxylate organic monomers, and Zn(II) imidazolates, are known to be stable in aqueous media, and may therefore be particularly useful in the invention.

Examples of preferred MOFs are the zirconium-based UiO-66, which comprises terephthalate (C₆H₄(COO)₂²⁻) organic monomers and cationic hexanuclear zirconium oxide clusters Zr₆O₄(OH)₄¹²⁺ as inorganic monomers, each carboxylate group of the terephthalates coordinated to two cations of the inorganic monomer, and the corresponding hafnium derivative of UiO-66, Hf-UiO-66, which has Hf₆O₄(OH)₄¹²⁺ oxide cluster inorganic monomers and terephthalate organic monomers. UiO-66, Hf-UiO-66, and MOFs having the same topology (network geometry), known as fcu network topology, comprise tetrahedral and octahedral pores, in a dynamic 2:1 ratio between the former and the latter. Each tetrahedral pore contains four corners where three organic linkers “meet”, i.e. extend towards the same spatial volume, and each octahedral pore contains six corners where four organic linkers “meet”. Free functional groups point toward the corner of the octahedral pore in the most stable conformation, but due to the dynamic rotation of the linker about its axis of connectivity and steric repulsion between neighbouring carboxylate groups, free functional groups may also frequently be directed into tetrahedral pores.

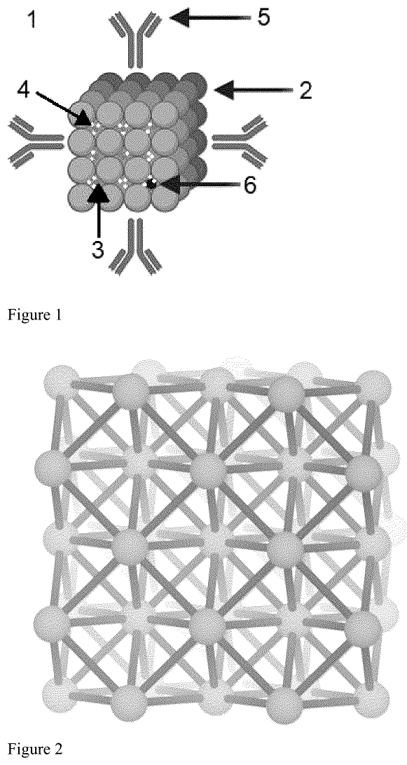

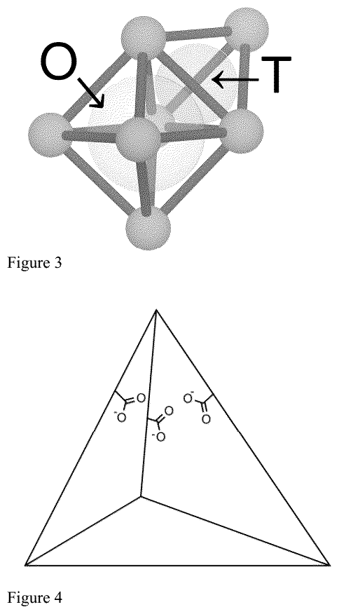

FIG. 2 shows a schematic representation of a network structure with fcu topology, where the inorganic monomers are shown as spheres with cuboctahedral coordination geometry, and the organic monomers are shown as bridging rods. Pores are illustrated in FIG. 3, which illustrates the octahedral (O) and tetrahedral pores (T) in fcu MOFs. The large transparent spheres illustrate the size of each of the pores.

M(IV)-based MOFs wherein M═Zr and/or Hf, i.e. MOFs wherein the inorganic monomers are based on metals Zr and/or Hf in their +4 (M(IV)) oxidation state, having carboxylate-based organic monomers, such as UiO-66, Hf-UiO-66, and derivatives thereof, may be particularly well-suited for in vivo applications for several reasons: (1) The monomers may have an advantageously low toxicity in vivo, (2) said MOFs may show an exceptional stability in aqueous solutions such as human serum, as shown in Experiments 2 and 3, owing to the stability of the M(IV)-carboxylate bond towards hydrolysis, and (3) the M(IV) oxocluster inorganic monomers may have a high connectivity, i.e. a high number of organic monomers connected to it such that free functional groups of several organic monomers may be spatially close to each other, which may allow the formation of highly coordinated chelating sites neighbouring the clusters. As a consequence, the MOF particles of the invention represent particularly stable and efficient carriers for radionuclides, which may have exceptionally high adsorption capacity enabling specific treatment with negligible leakage to other organs.

Mixed-metal and/or mixed-linker derivatives of UiO-66 can also be expected to show similarly advantageous properties as UiO-66. Relevant mixed-metal derivatives include derivatives comprising in the inorganic monomers two or more metals from the group comprising or consisting of Zr(IV) and Hf(IV). Relevant mixed-linker derivatives include derivatives having monomers known by the skilled person to have a coordinating geometry similar to terephthalate, such as monomers selected from the group comprising or consisting of aminoterephthalate, hydroxyterephthalate, mellitate, pyromellitate, sulfoterephthalate, muconate, and combinations thereof. Suitable mixed-linker MOFs can be obtained through synthesis where multiple linkers are present, or through linker exchange, which both are well-known to the person skilled in the art.

Further, MOFs having inorganic monomers based on Fe(III), Al(III), Ti(IV), and/or Cr(III) may also be particularly useful in the invention. Organic monomers may e.g. be selected from the group comprising or consisting of terephthalate, aminoterephthalate, hydroxyterephthalate, mellitate, pyromellitate, sulfoterephthalate, muconate, and combinations thereof. While said MOFs cannot form the same topology as UiO-66, they may form other interesting topologies that may function in the same manner.

The MOF of the invention comprises at least one free functional group that extends into a pore. Preferably, the MOF comprises a repeating three-dimensional network of inorganic monomers and organic monomers, forming pores, and at least one free functional group that extends into each pore, i.e. each pore has at least one free functional group extending into it. In some embodiments, only a certain amount of the pores has at least one free functional group extending into it, such as 50% of the pores, such as 80% of the pores, such as 95%, such as 100% of the pores.

The term “extending into a pore” as used herein means that the functional group is directed into, points into, and/or is present in one of the pores formed by the repeating three-dimensional network of inorganic monomers and organic monomers. Hence, the free functional group is available for coordinating to, binding and/or reacting with a compound, an ion, or the like, within the pore. In particular, the functional group is available for coordinating to, complexing, and/or binding a radionuclide, such as a radionuclide cation.

The at least one free functional group is connected to an inorganic monomer and/or to an organic monomer. When the at least one free functional group is connected to an organic monomer, it is preferably covalently bonded to the organic monomer. In some embodiments, each organic monomer of the MOF has at least one free functional group connected to it. In other embodiments, a predetermined amount of the organic monomers each has at least one free functional group connected to it. In some embodiments, each inorganic monomer of the MOF has at least one free functional group connected to it. In other embodiments, a predetermined amount of the inorganic monomers each has at least one free functional group connected to it.

The free functional group is preferably Lewis basic, and is preferably selected from the group comprising or consisting of carboxylic acid, carboxylate, hydroxyl, sulfonic acid, sulfonate, sulfhydryl, primary amines, secondary amines, and combinations thereof. The secondary amines are selected from the group comprising or consisting of amines of the general structure —NHR, wherein R is selected from the group comprising or consisting of C₁-C₈ alkyl, alkenyl, and alkynyl with at least one Lewis basic functional group (e.g. carboxylic acid, carboxylate, hydroxyl, sulfhydryl, sulfonic acid, sulfonate, primary amines, secondary amines, and combinations thereof), such as C₁-C₄ straight chain and branched alkyl, alkenyl, and alkynyl, with at least one Lewis basic functional group (e.g. carboxylic acid, carboxylate, hydroxyl, sulfonic acid, sulfonate, sulfhydryl, primary amines, secondary amines, and combinations thereof). Such functionality can for example be obtained by performing a peptide condensation of N-protected natural amino acids with free primary amino groups of the organic monomer of the MOF.

The free functional group or groups may advantageously be selected based on the choice of radionuclide. For example, if the radionuclide is known by the skilled person to be oxophilic, or “hard” within the HSAB theory (hard and soft (Lewis) acids and bases), the pores may advantageously comprise at least one oxygen-containing functional group, i.e. carboxylic acid, carboxylate, hydroxyl, sulfhydryl, sulfonic acid, and/or sulfonate. Correspondingly, if the radionuclide is “soft” within the HSAB theory, the pores may advantageously comprise at least one amine group. In some embodiments, the functional group or groups are further selected based on the daughters of the radionuclide, such as by using both at least one oxygen-containing functional group and at least one amine group if the radionuclide is hard and a daughter is soft, or vice versa.

Hence, in some embodiments, the MOF comprises as least one free functional group selected from the group comprising or consisting of carboxylic acid, carboxylate, hydroxyl, sulfonic acid, sulfonate, sulfhydryl, primary amines, secondary amines, and combinations thereof. In other embodiments, the MOF comprises as least one free functional group selected from the group comprising or consisting of carboxylic acid, carboxylate, hydroxyl, sulfonic acid, sulfhydryl, sulfonate, and combinations thereof. In yet other embodiments, the MOF comprises as least one free functional group selected from the group comprising or consisting of primary amines and secondary amines and combinations thereof.

Preferably, the MOF comprises 1-4 free functional groups, such as 1-2 functional groups, such as at least three, such as at least four free functional groups. In some embodiments, the MOF comprises as least two free functional groups, and the free functional groups are identical, or at least two, such as at least three, such as all, of the free functional groups are different from each other. In some embodiments, the MOF comprises as least three free functional groups, and the free functional groups are identical, or at least two, such as at least three, such as all, of the free functional groups are different from each other. In some embodiments, the MOF comprises as least four free functional groups, and the free functional groups are identical, or at least two, such as at least three, such as all, of the free functional groups are different from each other.

The MOF may be selected such that when the MOF comprises as least two free functional groups as disclosed above, the structure of the MOF allows some or all of the at least two free functional groups to point towards a common spatial volume, such as a common point or volume within the pore. The MOF may be selected such that when the MOF comprises as least two free functional groups as disclosed above, the structure of the MOF allows some or all of the at least two free functional groups to orient in a manner that, in the presence of a radionuclide cation, enables favourable interaction with the radionuclide cation, e.g. by aligning with outer orbitals of the radionuclide cation. In some embodiments, each free functional group has a distance of 4-15 Å from at least one, such as all, of the other free functional groups extending into the same pore, such as 4-12 Å, such as 5-10 Å.

Further, the choice and placement of the at least one free functional group may be used to modify the geometry of a coordinating, such as binding, such as chelating, site in a pore. Such site may be formed by the presence of one or more free functional groups, but may also result from the size and/or geometry of a pore. In some embodiments, such site is defined as comprising all free functional groups extending into the same pore. In some embodiments, such site is defined as comprising free functional groups extending into the same pore and that have a maximum distance from at least one other free functional group in the same pore that does not exceed 15 Å, such as 10 Å. In some embodiments, such a site is present in a corner of a pore. This placement may entail that the site is accessible from one direction only, thus limiting competitive absorption. Further, when such site is present in a corner of a pore, or in another way inside the pore as opposed to at the opening of the pore, other compounds, such as other cations, such as a competing cation, may be present in the pore and sterically block the radionuclide from leaving the pore. Such sites present in corners may in principle be constructed in all MOFs that can contain free functional groups. Examples include the MOFs Cr-MIL-100, Fe-MIL-100, and Al-MIL-100, Cr-MIL-53, Fe-MIL-53, and Al-MIL-53, Zr-MIL-140, Hf-MIL-140, Zr-MOF-711, Hf-MOF-711, ZIF-8, Ti-MIL-125.

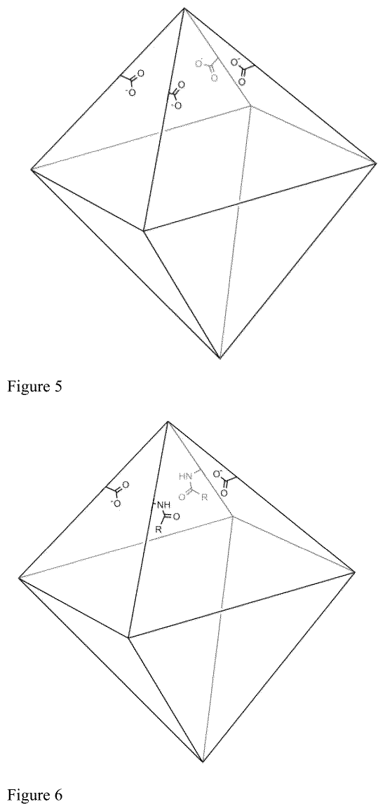

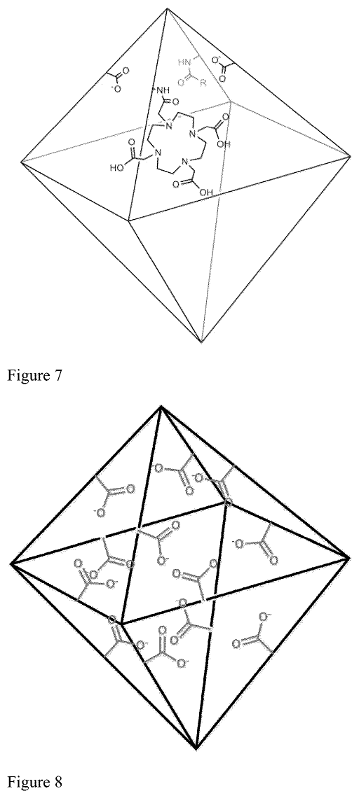

Examples of coordinating, binding and/or chelating sites are shown in FIGS. 4-7. FIG. 4 shows an example of a coordinating, binding and/or chelating site in a tetrahedral cage corner, provided by three free carboxylate groups directed into said pore. FIG. 5 shows an example of such a site in an octahedral pore corner, provided by four free carboxylate groups directed into said pore. FIG. 6 shows such a site in an octahedral pore corner, provided by two free carboxylate groups, and two free amido-groups directed into said pore. The latter can e.g. be obtained by postsynthetic peptide condensation on free primary amino groups. Postsynthetic modifications are discussed below. FIG. 7 shows such a site in an octahedral cage corner, provided by two free carboxylate groups, and two free amido-groups directed into said pore, where one amido-group is bearing a DOTA chelator. FIG. 8 shows a pore where several free functional groups are extending into the pore.

Further, the at least one functional group directed into the pores may be used to modify the charge balance of a pore and/or of a coordinating, such as binding, such as chelating, site in a pore. Advantageously, the MOFs of the invention comprise a free functional group, or a combination of functional groups, that result in a net negative charge in a pore. This net negative charge may facilitate strong, selective adsorption of cations.

The introduction of substituents on the phenyl ring of the terephthalate monomers of UiO-66 and the UiO-66-type MOFs gives easy access to crystalline MOFs comprising at least one free functional group extending into a pore, wherein the at least one free functional group is selected from the group comprising carboxylic acid, carboxylate, hydroxyl, sulfonic acid, sulfonate, sulfhydryl, primary amine, secondary amines, and combinations thereof. For example, UiO-66-COOH, UiO-66-(COOH)₂ and UiO-66-NH₂ are all commercially available. UiO-66-COOH, UiO-66-(COOH)₂, Hf-UiO-66-COOH, and Hf-UiO-66-(COOH)₂ represent preferred MOFs having 6-12 free carboxylic acid functional groups extending into their pores, whereof three or four free functional groups form a coordinating site in a pore, and that may thus chelate radionuclide cations in an efficient and/or stable manner.

Two main approaches exist for so-called pore engineering, i.e. introduction of free functional groups that extend into a pore. The synthesis of MOFs using “preintegrated organic monomers” in which two different coordinating sites coexist for framework construction and for other use, and “postsynthetic modification”, such as sequential attachment of further functional groups on preconstructed MOFs. Free functional groups in a MOF can be modified through chemical reactions, colloquially known as “postsynthetic modification”. In principle, all chemical reactions that can be performed by standard wet-chemical methods can also be performed on the same functional group in a MOF. For example, an amino acid can be reacted with a free primary amine group in a MOF by a peptide condensation reaction. Molecules that have specific properties can be grafted onto the MOF, such as within pores of the MOF, using this approach. Hence, MOFs having particular structures and particular free functional groups are available both by designing and synthesising new MOFs, and by modifying existing MOFs. Derivatives of various MOFs having the UiO-66 structure, as well as MOFs having other structures, and comprising various free functional groups, are thus readily available. In some embodiments, at least one radionuclide chelator known to the skilled person, such as EDTA, such as DOTA, is grafted onto a group extending into a pore, such that at least one free functional group is a known chelator.

Further, most MOFs can tolerate a certain fraction of linker vacancy defects, i.e. a site where an organic linker is missing from the structure, leaving coordinatively unsaturated sites on the adjacent inorganic monomer. These unsaturated sites can be modified with coordinating molecules, such as a molecule that can coordinate the inorganic monomer with at least one functional group and that has a free functional group for extending into a pore, e.g. an amino acid, that can contribute to the functional properties of the MOF.

Preferably, the MOF of the invention comprises a repeating three-dimensional network of inorganic monomers and organic monomers, forming pores, and at least one free carboxylic acid or carboxylate functional group that extends into each pore.

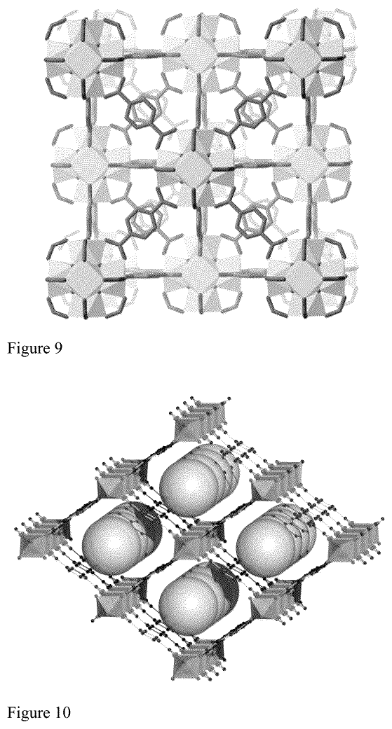

FIGS. 9, 10, and 11 show schematic representations of preferred MOFs.

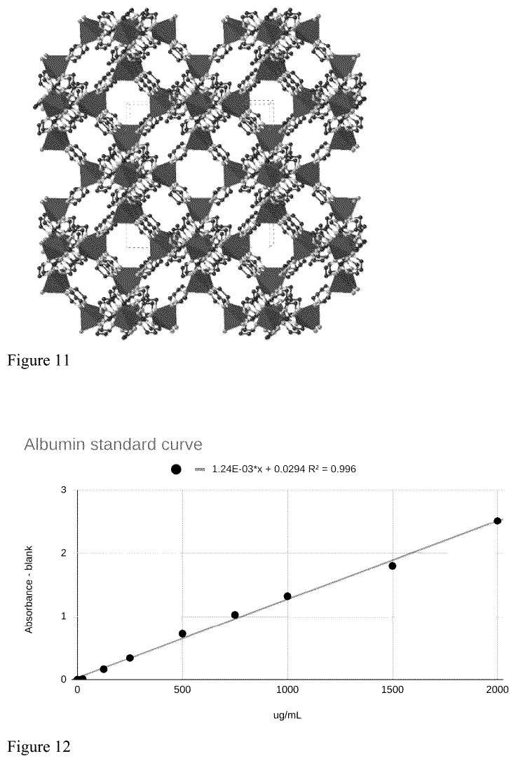

FIGS. 9 and 10 show partial crystal structures of M(IV) and M(III) based terephthalate MOFs. FIG. 9 illustrates UiO-66 (organic monomer shown as sticks where each CIO atom is situated on the corners, inorganic monomer as polyhedra). FIG. 10 illustrates MIL-53, where in addition to lines and polyhedra, the available pore space is illustrated by large spheres.

The preferred MOFs shown in FIGS. 9 and 10 may have M(IV) inorganic monomers. Suitable organic monomers are shown below (structures I, II, Ill, IV, V, and VI):

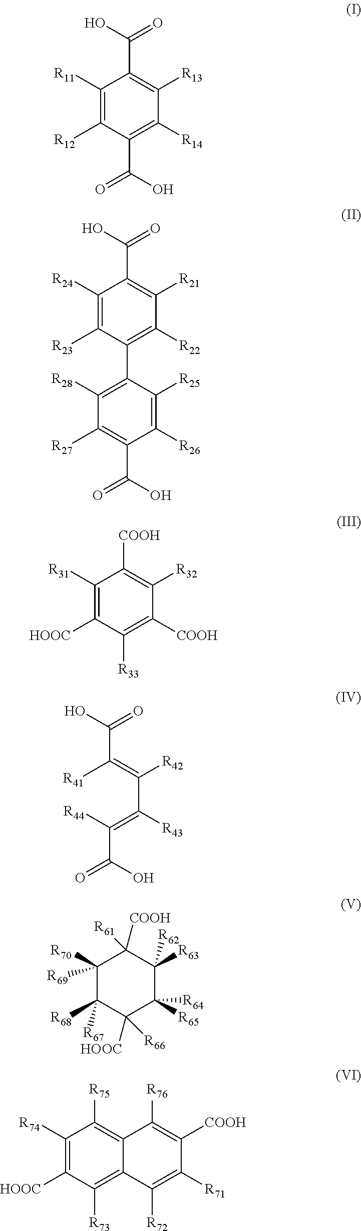

Structure I represents a terephthalic acid/terephtalate organic monomer, which may form MOFs with M(III) and M(IV) based inorganic monomers through metal-carboxylate bonds. R₁₁, R₁₂, R₁₃, and R₁₄ may each independently be selected from the group comprising or consisting of hydrogen, carboxylic acid, carbon/late, hydroxyl, sulfonic acid, sulfonate, sulfhydryl, primary amines, and secondary amines, i.e. each of the R groups may be hydrogen or a free functional group.

Structure II represents a biphenyl-4,4′-dicarboxylic acid organic monomer, which may form MOFs with M(III) and M(IV) based inorganic monomers through metal-carboxylate bonds. R₂₁, R₂₂, R₂₃, R₂₄, R₂₅, R₂₆, R₂₇, and R₂₈ may each independently be selected from the group comprising or consisting of hydrogen, carboxylic acid, carboxylate, hydroxyl, sulfonic acid, sulfonate, sulfhydryl, primary amines, and secondary amines, i.e. each of the R groups may be hydrogen or a free functional group.

Structure III represents a trimesic acid (1,3,5-benzenetricarboxylic acid) organic monomer, which may form MOFs with M(III) and M(IV) based inorganic monomers through metal-carboxylate bonds. R₃₁, R₃₂, and R₃₃ may each independently be selected from the group comprising or consisting of hydrogen, carboxylic acid, carboxylate, hydroxyl, sulfonic acid, sulfonate, sulfhydryl, primary amines, and secondary amines, i.e. each of the R groups may be hydrogen or a free functional group.

Structure IV represents an adipic acid organic monomer, which may form MOFs with M(III) and M(IV) based inorganic monomers through metal-carboxylate bonds. R₄₁, R₄₂, R₄₃, and R₄₄ may each independently be selected from the group comprising or consisting of hydrogen, carboxylic acid, carboxylate, hydroxyl, sulfonic acid, sulfonate, sulfhydryl, primary amines, and secondary amines, i.e. each of the R groups may be hydrogen or a free functional group.

Structure V represents a 1,4-cyclohexyldicarboxylic acid organic monomer, which may form MOFs with M(III) and M(IV) based inorganic monomers through metal-carboxylate bonds. R₆₁, R₆₂, R₆₃, R₆₄, R₆₅, R₆₆, R₆₇, R₆₈, R₆₉, and R₇₀ may each independently be selected from the group comprising or consisting of hydrogen, carboxylic acid, carboxylate, hydroxyl, sulfonic acid, sulfonate, sulfhydryl, primary amines, and secondary amines, i.e. each of the R groups may be hydrogen or a free functional group.

Structure VI represents a naphthyl organic monomer, which may form MOFs with M(III) and M(IV) based inorganic monomers through metal-carboxylate bonds. R₇₁, R₇₂, R₇₃, R₇₄, R₇₅, and R₇₆ may each independently be selected from the group comprising or consisting of hydrogen, carboxylic acid, carboxylate, hydroxyl, sulfonic acid, sulfonate, sulfhydryl, primary amines, and secondary amines, i.e. each of the R groups may be hydrogen or a free functional group.

In each of structures I-VI, the secondary amines may be selected from the group comprising or consisting of amines of the general structure —NHR, wherein R is selected from the group comprising or consisting of C₁-C₈ alkyl, alkenyl, and alkynyl with at least one Lewis basic functional group (e.g. carboxylic acid, carboxylate, hydroxyl, sulfhydryl, sulfonic acid, sulfonate, sulfhydryl, primary amines, secondary amines, and combinations thereof), such as C₁-C₄ straight chain and branched alkyl, alkenyl, and alkynyl, with at least one Lewis basic functional group (e.g. carboxylic acid, carboxylate, hydroxyl, sulfhydryl, sulfonic acid, sulfonate, sulfhydryl, primary amines, secondary amines, and combinations thereof).

FIG. 11 illustrates MOFs having a zeolitic imidazolate framework and a so-called sod topology. The organic monomer is shown as balls and sticks to represent atoms and bonds, and Zn atoms are shown as tetrahedra.

The preferred MOFs shown in FIG. 11 may have MOO inorganic monomers. Suitable organic monomers are shown below (structure VII):

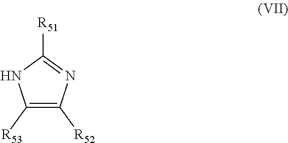

Structure VII represents a imidazole organic monomer, which may form MOFs (also known as ZIFs) with MOO based inorganic monomers through metal-imide bonds. R₅₁, R₅₂, and R₅₃ may each independently be selected from the group comprising or consisting of hydrogen, carboxylic acid, carboxylate, hydroxyl, sulfonic acid, sulfonate, sulfhydryl, primary amines, and secondary amines, i.e. each of the R groups may be hydrogen or a free functional group. The secondary amines may be selected from the group comprising or consisting of amines of the general structure —NHR, wherein R is selected from the group comprising or consisting of C₁-C₈ alkyl, alkenyl, and alkynyl with at least one Lewis basic functional group (e.g. carboxylic acid, carboxylate, hydroxyl, sulfhydryl, sulfonic acid, sulfonate, primary amines, secondary amines, and combinations thereof), such as C₁-C₄ straight chain and branched alkyl, alkenyl, and alkynyl, with at least one Lewis basic functional group (e.g. carboxylic acid, carboxylate, hydroxyl, sulfhydryl, sulfonic acid, sulfonate, primary amines, secondary amines, and combinations thereof).

In the invention disclosed herein, the MOF is present in the form of a particle, such as a microparticle or a nanoparticle. Preferably, the particle is a nanoparticle. As used herein, the term “nanoparticle” refers to any particle having a diameter of less than 1000 nm, such as from 1 to 1000 nm. Similarly, the term “nanoparticles” refers to a plurality of particles having an average diameter of between about 1 and 1000 nm. Reference to the “size” of a nanoparticle is a reference to the length of the largest straight dimension of the nanoparticle. For example, the size of a perfectly spherical nanoparticle is its diameter. The size may e.g. refer to the hydrodynamic radius of the particle characterized by dynamic light scattering. In some embodiments, the nanoparticles of the invention have a particle size of 1-200 nm. Correspondingly, the term “microparticle” refers to any particle having a diameter of less than 1000 μm, such as from 1 to 1000 μm.

Micro and nanoparticles have found some use in radiotherapy. An important factor when such particles are to be used is their stability, both in the sense that the particles themselves should be completely stable or slowly degradable, in order to reduce the risk of system toxicity, and in the sense that the radionuclide should strongly associated to the particle to avoid leakage of the radionuclide. The MOFs of the invention are very stable, as discovered by the inventors and illustrated in Examples 2 and 3. Further, the presence of the free functional groups, along with the confining effect of the pores themselves, ensure a strong chelation of a radionuclide to the particle as illustrated in example 7. The choice of free functional groups may also lead to a high level of selectivity, e.g. in that cations rather than anions, and/or hard rather than soft ions, are preferably chelated. Hence, the invention provides particularly stable carriers for radionuclides.

Throughout a MOF particle of the invention, 10\hat{ }3 to 10\hat{ }5 pores exist, with the possibility of trapping a radionuclide cation in a fraction of them, i.e. the fraction accessible from the outside of the particle. Should a radionuclide cation be released from one pore, it may be caught by a neighbouring pore, preventing leakage from the particle. This ability of “re-adsorbtion” of desorbed radionuclide cations indicates that the MOF particles of the invention may more efficiently hold on to the radionuclide cations compared to conventional chelators. Further, the number of cations per particle can be tuned, thus tuning the level of radioactivity of the particle.

Due to the presence of pores having a suitable diffusion sphere and the free functional groups extending into the pores, if a particle according to the invention is exposed to a solution comprising radionuclide cations, these cations will be readily adsorbed and chelated within the pores. An additional advantage of using a MOF particle according to the invention is that any competitive species, such as any other cations present in the solution, may occupy vacant pores rather than evicting the radionuclide cation.

The MOFs of the invention, as well as the particles thereof, may be obtained by any manner known to the skilled person, such as obtained commercially, such as synthesised using any synthetic protocol available to the skilled person.

In some embodiments, the particle of the invention comprises at least one targeting moiety connected to the particle on an external surface of the particle. The term “external surface” as used herein with respect to a particle, refers to the surfaces at its exterior, as opposed to the surface of a pore within the particle. Where pores permeate a particle and are visible within it, the external surface is defined to include all surfaces at the outermost faces of the particle, but not surfaces that define the pores.

As used herein, the term “targeting moiety” refers to a moiety, such as a molecule, such as a part of a molecule, which is “tissue targeting”, i.e. that serves to localise itself—and any moiety, such as a particle, to which it is connected—preferentially to at least one tissue site at which its presence, e.g. to deliver a radioactive decay, is desired. The term “targeting moiety” can also refer to a functional group that serves to target or direct a particle to a particular location, cell type, diseased tissue, or association. The targeting moiety may, for example, be a moiety known by the skilled person to bind to or complex with a biomarker, such as a cell-surface marker, e.g. a receptor, transport protein, cell adhesion molecule, present on a cell affected by a disease, or cells in the vicinity of such cells. Such cell-surface markers include, but are not limited to, proteins more heavily expressed on diseased cell surfaces than on healthy cell surfaces or those more heavily expressed on cell surfaces during periods of growth or replication of cells than during dormant phases. Components present in the vicinity of target cells or tissues or associated therewith may also be utilised at the target for therapy in accordance with any aspect of the invention. For example, components present in or released into the matrix around targeted cells or tissues may be used for targeting if the presence, form or concentration allows the region to be distinguished from healthy tissue.

Non-limiting examples of targeting moieties include
small molecule targeting moieties, such as folic acid; folate; carbohydrates; monosaccharides such as glucose, mannose, galactose; urea derivatives; lipids; streptavidin; albumin; biotin; steroidal and non-steroidal hormones;
aptamers, i.e. single-stranded oligonucleotides that recognise specific binding domains of a receptor;
macromolecules, such as biomacromolecules, such as antibodies; peptides, such as biomimetic peptides, such as phage display peptides; proteins; nucleic acid; and
cells, such as natural cells, such as genetically engineered cells.

Preferred targeting moieties include antibodies, antibody fragments, antibody constructs, constructs of antibody fragments, minibodies, nanobodies, intrabodies, unibodies, affibodies, and diabodies. As used herein, the term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. The term “antibody” encompasses e.g. monoclonal, polyclonal, recombinant, humanized, and/or chimeric antibodies. The antibody may be a member of any immunoglobulin class, including any of the human classes: IgG, IgM, IgA, IgD, and IgE. Examples of useful antibodies are conventional full length antibodies (e.g IgG) and camelid heavy chain antibodies (VHH).

As used herein, the term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)₂, scFv, Fv, dsFv diabody, and Fd fragments.

Cell-based targeting, although in its early-stage of development, may hold advantages including high specificity and versatility. Therapeutics can be either coupled to cell surfaces or encapsulated within cells. Many cell types, including red blood cells, leukocytes, stem cells, platelets, dendritic cells, and even bacteria, have been leveraged for tissue-specific delivery of small molecules, large molecules, and even nanoparticles. When a cell is used as targeting moiety, the treatment of the invention can be viewed as cell therapy. Use of particles of the invention comprising cells, e.g CAR/TCR T cells, CAR/TCR NK cells, NK-92, as targeting moieties, for allo- or autologous cell therapy can have both potent tumour killing effect, and immunomodulatory effects preventing detrimental side effects of cell therapy (e.g cytokine release syndrome, graft vs host disease).

In some embodiments, the targeting moiety is one of two or more components collectively having the effect of targeting the particle of the invention to the desired tissue(s). This may be, for example, where one component binds to a particular tissue, tumour, or cell-type (a tissue-binding agent) and a second or further component, the targeting moiety, binds to the tissue-binding agent. Suitable specific binding pairs for providing the tissue binding agent and targeting moiety with mutual affinity are known in the art (e.g. biotin with avidin or streptavidin).

The targeting moiety may be obtained by any manner known to the skilled person, such as obtained commercially, such as synthesised using any synthetic protocol available to the skilled person, such as enzymatically, synthetically and/or chemically produced.

In some embodiments, the particle comprises only one targeting moiety. In other embodiments, the particle comprises two or more targeting moieties, such as five, such as ten targeting moieties. The number of targeting moieties may be selected based on the size of the particle. The targeting moieties may be the same or different from each other. The mass ratio of targeting moiety and particle may depend on the molecular weight of the targeting moiety and the diameter of the particle.

The use of two or more different targeting moieties may have the advantage of improved binding to tissue or cells expressing a profile of antigens associated with the target (e.g M2 tumour associated macrophages expressing CD163 and CD206), since more than one antigen can be targeted. An additional advantage may be the ability to target vessels that accumulate at the tumour site (e.g. VCAM1) as well as a tumour specific antigen.

The at least one targeting moiety may be linked directly to the particle, such as via a covalent bond, or the at least one targeting moiety may be linked to the particle via a linking group. Target moiety can also be linked to the particles covalently conjugated to streptavidin which strongly bind biotin/biotin derivates. Streptavidin may then function as a linker, but also as a binding moiety for biotinylated moieties. Thus, streptavidin conjugated particles can be combined with one or more biotinylated targeting moieties. Methods for linking a targeting moiety to a surface are well-known in the art; standard organic and/or inorganic chemistry may be used, such as carbodiimide coupling. Suitable linking groups may readily be determined by the person skilled in the art; for example, a range of linking groups are known from the field of antibody-drug-conjugates. Non-limiting examples of linking groups include poly(ethylene glycol) (PEG), 2-(maleimidomethyl)-1,3-dioxanes (MD), and maleimidocaproyl succinimidyl 4-(N-maleimidomethyl)cyclohexane-1-carboxylate) (SMCC). The external surface of a MOF particle may comprise the same organic functional groups as the interior of the pores, which may be subject to the same reactions. However, size discrimination may be used in order to perform reactions on the external surface and not in the pores by using reagents that are sterically prohibited from entering the pores of the MOF, thus enabling selective attachment of targeting moieties to the external surface of the particle.

The at least one targeting moiety may be a targeting moiety for targeting a cell affected by a proliferative disease, such as tumour cell, a cancer cell, a cell affected by a hyperplastic disease, a cell affected by a neoplastic disease. In preferred embodiments, the targeting moiety is a targeting moiety for targeting a cancer cell. As used herein, the terms “cancer cell” and “tumour cell” refer to cells that divide at an abnormal, increased rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumours of the nervous system including glioma, glioblastoma multiform, meningoma, medulloblastoma, schwannoma and epidymoma. The person skilled in the art is knowledgeable about targeting moieties that may be used for targeting specific types of cells affected by proliferative diseases, in particular cancer cells.

In another embodiment, the particle of the invention comprises no targeting moiety. Whether a drug substance is administered into the human body through the oral, intravenous or parenteral route, they will be metabolized by the hepatic portal system (liver) which is the site for most metabolism, by metabolizing enzymes, which will degrade the substances delivered for easy removal from the body. Accordingly, the particles of the invention, where no targeting moiety is included, will after having been administrated to a subject be transported to the liver, and could be well suited for therapy of the liver. Furthermore, the natural biodistribution of the particle can also be used to obtain radioisotopes to the given site and radiate the tissue in situ.

The MOF particle of the invention comprises at least one radionuclide. As used herein, the term “radionuclide”, which may also be referred to as a radioactive nuclide, radioisotope or radioactive isotope, is an atom that has excess nuclear energy, making it unstable. This excess energy can be used in one of three ways: emitted from the nucleus as gamma radiation; transferred to one of its electrons to release it as a conversion electron; or used to create and emit a new particle (alpha particle or beta particle) from the nucleus. During those processes, the radionuclide is said to undergo radioactive decay. The resulting nuclide is referred to as a daughter or as progeny.

The at least one radionuclide is located in a pore, such as present in a pore, such as enclosed by a pore, preferably coordinated to and/or chelated by the at least one free functional group extending into said pore. Preferably, the radionuclide is a radionuclide cation. More preferably, the radionuclide is a radionuclide selected from the group comprising or consisting of radium-223, radium-224, radium-225, bismuth-212, bismuth-213, lead-212, actinium-225, terbium-149, terbium-161 and thorium-227. In some embodiments, the radionuclide is a radionuclide cation wherein the radionuclide is selected from the group comprising or consisting of radium-223, radium-224, radium-225, bismuth-212, bismuth-213, lead-212, and thorium-227 Preferably, the radionuclide is radium-223 or radium-224, more preferably the radionuclide is radium-223.

The radionuclides listed above have a half-life of 1-20 days, compatible with tumour killing and clinical administration, and thus have a great therapeutic potential, described below for each isotope. These radionuclide cations listed above may bind strongly to negatively charged chelating groups of the MOFs, such that radioisotope leakage, which otherwise occurs due to competition with salts and other components in vivo, may be limited. Daughter isotopes of these radioisotopes may also be captured and retained in these MOFs.

Radium-223, 224 and 225:

Radium has a long history in treatment of cancer. All radium isotopes are radioactive, but it is only Ra-223 (half-life (t½)=11.4 days), Ra-224 (t½=3.6 days) and Ra-225 (t½=14.9 days) that have half-lives compatible with use as therapeutic radionuclides. They all decay via multiple α- and β-emitting progeny with shorter half-lives than their respective radium parent, with an average of four emitted α-particles per 17 introduction complete decay. The complete decay of each series releases a high total energy of 28-29 MeV, where more than 90% of the energy is associated with the α-emissions. The three radium isotopes mentioned above can all be obtained from their long-lived parental radionuclides: Ra-223 from Ac-227 (t½=21.8 years), Ra-224 from Th-228 and Ra-225 from Th-229. In the same way as Th-228, Ac-227 can be produced by neutron irradiation of natural Ra-226, but it is also available in uranium minerals from decay of U-235.

Radium has only one oxidation state (+II), and due to the very alkaline nature of Ra2+ cations in aqueous solutions, radium is not easily complexed. Thus, most radium compounds are simple ionic salts. This inherent property has made it problematic to couple radium to targeting molecules. The main obstacle with Ra-223 and Ra-224 is thus not related to production or availability, as is the case for several of the other α-emitters.

Thorium-227:

Thorium-227 has a half-life of 18.7 days and releases five α-particles during its decay to stable 207Pb. It is the immediate parent radionuclide of Ra-223, and highly purified Th-227 can be obtained from the same Ac-227 generator used for production of Ra-223. Th-227-complexes will be challenged by recoil energy from the α-emitting daughters that can lead to their release and subsequent redistribution. Both the metabolic processing of a complex and recoil energy during decay can lead to release of progeny, which may raise toxicity concerns and limit the dose that can be administered. Decay occurring in non-targeted tissues is also a therapeutic disadvantage because the full arsenal of α-particles does not contribute to the radiation dose delivered to the tumour. The particles of this invention may be able to bind Th-227 along with its daughter isotopes.

Bismuth-212 and bismuth-213:

The bismuth isotopes of interest for α-therapy, Bi-212 and Bi-213, have short half-lives of 60.6 minutes and 45.6 minutes, respectively. They are the final α-emitting daughters in the Ra-224 and Ac-225Ac decay chains and can thus be eluted from generators based on these respective radionuclides.

Lead-212:

Lead-212 has a half-life of 10.6 h and decays via 6-emission to the therapeutically potent α-emitting daughter Bi-212. For that reason, 212Pb can be used as an in vivo generator of α-particles from Bi-212, resulting in a virtual prolongation of the Bi-212 half-life. Lead-212 is available from generators based on Ra-224 and the half-life necessitates on-site production.

Actinium-225:

The radiometal 225Ac has a half-life of 10.0 days and generates an average of four α-particles per complete decay to stable 209Bi. It can be obtained by radiochemical extraction from 229Th (t½=7340 years) or by different accelerator-based methods. The use of 225Ac for radiolabelling of targeting molecules has been limited by lack of appropriate chelators, both to give sufficient yield and stability.

Terbium-149:

Terbium-149 (Tb149) has a half-life of 4.12 days and emits alpha and beta particles in addition to gamma rays. Tb149 can be produced by gadolinium Gd152(p,4n) reaction, or by different accelerator-based methods.

Terbium-161:

Terbium-161 (Tb161) has a half-life of 6.9 days and can be produced in a reactor by neutron bombardment on enriched gadolinium 161 (Gd161) target to produce Gd161 which subsequently decays to Tg161 or by different accelerator-based methods.

Like the other radioisotope described, Gd161 generally occurs as a cation (3+) and is well suited as an isotope for RIT. However, it is a beta-emitter which results in a lower LET and longer penetration length in the tissue, which can be an advantage to penetrate deeper into larger non-vascularised tumours.

In some embodiments, the particle comprises one radionuclide. In other embodiments, the particle comprises more than one radionuclide, such as two, such as three, such as five radionuclides, independently selected from the group comprising radium-223, radium-224, radium-225, bismuth-212, bismuth-213, lead-212, actinium-225, and thorium-227, preferably a radionuclide cation wherein the radionuclide is selected from radium-223 or radium-224, more preferably being radium-223. If the particle comprises more than one radionuclide, the radionuclides will typically be located in separate pores, although the presence of two radionuclides in the same pore may also be possible. It should be noted that in all embodiments, complete control of the number of radionuclides per particle cannot be expected; during a chelation process wherein particles are contacted with a solution containing radionuclides, a statistical distribution of radionuclides per particle will result.

The particle disclosed herein, i.e. a particle comprising a MOF,

wherein the MOF comprises a repeating three-dimensional network of inorganic monomers and organic monomers, forming pores, and at least one free functional group that extends into a pore, wherein the functional group is selected from the group comprising carboxylic acid, carboxylate, hydroxyl, sulfonic acid, sulfonate, sulfhydryl, primary amines, secondary amines, and combinations thereof, wherein the particle optionally further comprises at least one targeting moiety connected to the particle on an external surface of the particle, and at least one radionuclide, wherein the at least one radionuclide is selected from the group comprising radium-223, radium-224, radium-225, bismuth-212, bismuth-213, lead-212, actinium-225, thorium-227, terbium-149 and terbium-161 and wherein the at least one radionuclide is located in at least one of the pores, may have at least the following advantages compared to the state of the art:
(a) In contrast to existing small-molecule chelators, the MOF particle contains numerous chelating sites, which may enable dose control by loading the particles with a predetermined number of radionuclides.
(b) The network structure of the MOF may positively affect the ability of the particle to contain the radiation, such as by the following mechanisms:
   (1) In contrast to state-of-the-art molecular chelators, the MOF may be able, through physisorption, to contain daughter isotopes that appear through the decay chains of a radionuclide, thus keeping also the daughters close to the tumour cells,
   (2) In contrast to state-of-the-art molecular chelators, adsorbed radionuclides in MOFs may be less susceptible to competitive adsorption by other ions because these may be adsorbed by vacant pores, and
   (3) desorbed radionuclides may be re-adsorbed by adjacent, vacant pores.

In summary, the invention may thus provide an improved method of radiotherapy.

Regarding (b)(2), competitive adsorption may be relevant not only during preparation and storage of the particles and during adsorption of the radionuclide cation, but also when the particle is used as a drug. For instance, Ra²⁺ may, on a general basis, be susceptible to competition from Mg²⁺ and Ca²⁺—but less so using a MOF than using a small-molecule chelator. Reference is made to Example 7, showing that a MOF of the invention, UiO-66-(COOH)₂ absorbed Ra223 very well, and retained this over several days.

The particle of the invention may further comprise one or more molecules for modifying the external surface of the particle. Such one or more molecules may be connected to, such as covalently bound to, such as coordinated to, the particle on an external surface of the particle. Again, size discrimination may be used in order to perform reactions on the external surface and not in the pores. For example, the exterior surface of a MOF comprising free carboxyl or amino groups can be functionalised with one or more compounds selected from the group comprising or consisting of PEG derivatives, N-hydroxysuccinimide (NHS), N-hydroxysulfosuccinimide (sulfo-NHS), MD linker, Mal-PAB, albumin, to facilitate transport, prevent agglomeration and/or provide targeting functionality by increasing the targeting flexibility and spatial distance between a MOF and its target. Albumin can be used to prevent agglomeration and to increase the blood half-life of the particles.

The particle of the invention may further comprise one or more further compounds, such as a molecule, such as an ion, located in at least one of the pores. Non-limiting examples include buffers and/or particular ions, such as ions for limiting leakage of radionuclide by steric blockage.

The MOF particles are typically produced by an aqueous synthesis at 20-100C.° where a solution of a suitable inorganic precursor for the inorganic monomer and a solution of the organic monomer are mixed. In some cases, the organic monomer is dissolved directly in the solution of the inorganic precursor or vice versa. In some cases, a growth controlling factor (also known as growth modulator) is added to affect the particle growth rate. The MOF particles precipitate from the solution as the structure is formed. Published procedures for the production of MOFs may be followed.

Targeting moieties may be attached, linked, bonded—generally referred to as conjugated—to the MOF particles directly, or via a linker. As an example: carboxyl MOFs can be conjugated to amine groups (present on the targeting unit) by carbodiimid (EDC, NHS/Sulfo-NHS) based cross linking.

Radiolabelling may be performed by mixing a solution or a suspension of the radionuclide cation homogeneously with a suspension of the particles for >1 minutes, and then separating residual unbound radionuclide cation from the labelled particles, such as by centrifugation or column purification. The radiolabelling procedure may be more convenient, such as in terms of time used, purification of the product, etc., if the targeting moiety is present on the particle before radiolabelling takes place.

Hence, the particle of the invention may be prepared by providing a particle, such as a nanoparticle, comprising a MOF,

wherein the MOF comprises
a repeating three-dimensional network of inorganic monomers and organic monomers, forming pores, and
at least one free functional group that extends into a pore, wherein the functional group is selected from the group comprising carboxylic acid, carboxylate, hydroxyl, sulfonic acid, sulfonate, sulfhydryl, primary amines, secondary amines, and combinations thereof,

wherein the particle optionally comprises
at least one targeting moiety connected to the particle on an external surface of the particle,

and contacting the particle with a radionuclide, such as a radionuclide cation, wherein the radionuclide is selected from the group comprising radium-223, radium-224, radium-225, bismuth-212, bismuth-213, lead-212, actinium-225, thorium-227, terbium-149 and terbium-161.

The radionuclide cation may, for example, be present in composition comprising a liquid.

In another aspect, the invention relates to a kit comprising,

in a first container,

a particle comprising
a MOF, wherein the MOF comprises
   a repeating three-dimensional network of inorganic monomers and organic monomers, forming pores, and
   at least one free functional group that extends into a pore, wherein the functional group is selected from the group comprising carboxylic acid, carboxylate, hydroxyl, sulfonic acid, sulfonate, sulfhydryl, primary amines, secondary amines, and combinations thereof;
optionally at least one targeting moiety connected to the particle on an external surface of the particle; and

and in a second container,

a radionuclide, wherein the radionuclide is selected from the group comprising radium-223, radium-224, radium-225, bismuth-212, bismuth-213, lead-212, actinium-225, thorium-227, terbium-149 and terbium-161 or a radionuclide generating any one of radium-223, radium-224, radium-225, bismuth-212, bismuth-213, lead-212, actinium-225, thorium-227, terbium-149 and terbium-161 as a daughter.

The skilled person will appreciate that the first and the second container may further comprise liquids, such as solvents, such as for dissolving or suspending any component, as well as further components such as at least one carrier, diluent, and/or excipient.

The particle disclosed herein may be present as an active ingredient in a desired dosage unit formulation, such as a pharmaceutically acceptable composition containing a conventional pharmaceutically acceptable carrier. As used herein, the term “pharmaceutically acceptable” means that compound must be physiologically acceptable to the recipient as well as, if part of a composition, compatible with other ingredients of the composition. The term “composition” refers to a mixture, in any formulation, of one or more compounds according to the invention with one or more additional chemical component.

Thus, in another aspect, the invention relates to a composition comprising at least one particle, wherein the at least one particle comprises a MOF,

wherein the MOF comprises
a repeating three-dimensional network of inorganic monomers and organic monomers, forming pores, and
at least one free functional group that extends into a pore, wherein the functional group is selected from the group comprising carboxylic acid, carboxylate, hydroxyl, sulfonic acid, sulfonate, sulfhydryl, primary amines, secondary amines, and combinations thereof,

wherein the particle comprises
optionally at least one targeting moiety connected to the particle on an external surface of the particle, and
at least one radionuclide, wherein the at least one radionuclide is selected from the group comprising radium-223, radium-224, radium-225, bismuth-212, bismuth-213, lead-212, actinium-225, thorium-227, terbium-149 and terbium-161,

and wherein the at least one radionuclide is located in at least one of the pores, together with at least one pharmaceutically acceptable carrier, diluent, and/or excipient.

The particle is defined as disclosed above. It may be a microparticle or a nanoparticle. Preferably, the particle is a nanoparticle.

The composition may be considered to be a pharmaceutical composition, as it comprises an active agent, i.e. the particle, combined with at least one pharmaceutically acceptable carrier, diluent, and/or excipient, making the composition especially suitable for therapeutic use.

The composition preferably comprises a multitude of the particles of the invention. The particles can be the same of different, i.e. with regards to type and number of radionuclides and/or targeting moieties. In some embodiments, the composition is a particle suspension comprising monodisperse or polydisperse particles labelled with a radionuclide cation.

The composition may further include one or more of any conventional, pharmaceutically acceptable excipients and/or carriers, e.g. solvents, fillers, diluents, binders, lubricants, glidants, viscosity modifiers, surfactants, dispersing agents, disintegration agents, emulsifying agents, wetting agents, suspending agents, thickeners, buffers, pH modifiers, absorption-delaying agents, stabilisers, antioxidants, preservatives, antimicrobial agents, antibacterial agents, antifungal agents, chelating agents, adjuvants, sweeteners, aromas, and colouring agents. Conventional formulation techniques known in the art, e.g., conventional mixing, dissolving, suspending, granulating, drageemaking, levigating, emulsifying, encapsulating, entrapping or compressing processes, may be used to formulate the composition.

In some embodiments, the composition is formulated for a particular method of administration to a subject.

The amount of particles according to the invention present in the composition can vary. In some embodiments, the amount of particles according to the invention present in the composition is 0.1-50% by weight, such as 1-30%, such as 50-20%. In other embodiments, the amount of the particles according to the invention present in the composition is 30-70% by weight, such as 40-60%. In yet other embodiments, the amount of the particles according to the invention present in the composition is 50-100% by weight, such as 50-70%, such as 50-80%, such as 60-98%, such as 70-95%.

The composition may also comprise MOF particles that do not comprise a radionuclide cation, such as particles comprising a MOF, wherein the MOF comprises a repeating three-dimensional network of inorganic monomers and organic monomers, forming pores, and at least one free functional group that extends into a pore, wherein the functional group is selected from the group comprising carboxylic acid, carbon/late, hydroxyl, sulfonic acid, sulfonate, sulfhydryl, primary amines, secondary amines, and combinations thereof, wherein the particle comprises at least one targeting moiety connected to the particle on an external surface of the particle. Such particles may be the same as the particles of the invention except for the absence of the radionuclide cation, or they may be different. The ratio between particles that comprise a radionuclide cation and particles that do not comprise a radionuclide cation may vary. In preferred embodiments, at least 90 of the particles comprise a radionuclide cation. The activity per mg particles may typically range from 10 kBq to 570 000 MBq, e.g. from 100 kBq to 570 000 MBq, or from 100 kBq to 1000 MBq. This corresponds to a maximum of 5% mass ratio (percentage radioisotope per MOF) using radium-223 as an example.

Further, the composition is substantially free of contaminants or impurities. In some embodiments, the level of contaminants or impurities other than residual solvent in the composition is below about 5% relative to the combined weight of the particles according to the invention and the intended other ingredients. In certain embodiments, the level of contaminants or impurities other than residual solvent in the composition is no more than about 2% or 1% relative to the combined weight of the particles according to the invention and the intended other ingredients.

In certain embodiments, the particle or composition according to the invention is sterile. Sterilisation can be achieved by any suitable method, including but not limited to by applying heat, chemicals, irradiation, high pressure, filtration, or combinations thereof.

The particle of the invention may be included in a composition and may be used as a medicament. Accordingly, in another aspect, the invention relates to a particle comprising
a MOF, wherein the MOF comprises
   a repeating three-dimensional network of inorganic monomers and organic monomers, forming pores, and
at least one free functional group that extends into a pore, wherein the functional group is selected from the group comprising carboxylic acid, carboxylate, hydroxyl, sulfonic acid, sulfonate, sulfhydryl, primary amines, secondary amines, and combinations thereof; and
optionally at least one targeting moiety connected to the particle on an external surface of the particle; and
at least one radionuclide, wherein the at least one radionuclide is selected from the group comprising radium-223, radium-224, radium-225, bismuth-212, bismuth-213, lead-212, actinium-225, thorium-227, terbium-149 and terbium-161:

and wherein the at least one radionuclide is located in at least one of the pores, or a composition comprising said particle together with at least one pharmaceutically acceptable carrier, diluent, and/or excipient,

for use as a medicament.

The particles of the invention, and compositions comprising said particles, may be used therapeutically, such for targeted delivery of radioactive decay to one or more particular sites in vivo, such as a particular cell, tissue, organ, etc. The targeting moieties target the particle to a particular site where radioactive decay, such as emission of an alpha particle, a beta particle, and/or a gamma particle, is desired, and the MOF serves as a chelating agent with the advantageous properties discussed above. When the particles do not comprise a targeting moiety, the particles will be transported to other organs for example the liver depending on the nanoparticles biodistribution properties, wherein the radioactivity will decay. Hence in one embodiment, the particles may be used therapeutically for treatment of indications where the nanoparticles naturally transport, particularly liver cancer.

For the various aspects of the invention as described herein that relate to use as a medicament and/or treatment of disease, particularly for the selective targeting of diseased tissue, the diseased tissue may in all embodiments reside at a single site in the body (for example in the case of a localised solid tumour) or may reside at a plurality of sites (for example in the case of a distributed or metastasised cancerous disease).

Due to the cytotoxic effects of radioactive decay, the particles and the compositions of the invention may be particularly useful against proliferative diseases. Thus, in a further aspect, the invention relates to a particle comprising
a MOF, wherein the MOF comprises
   a repeating three-dimensional network of inorganic monomers and organic monomers, forming pores, and
   at least one free functional group that extends into a pore, wherein the functional group is selected from the group comprising carboxylic acid, carboxylate, hydroxyl, sulfonic acid, sulfonate, sulfhydryl, primary amines, secondary amines, and combinations thereof;
optionally, at least one targeting moiety connected to the particle on an external surface of the particle; and
at least one radionuclide, wherein the at least one radionuclide is selected from the group comprising radium-223, radium-224, radium-225, bismuth-212, bismuth-213, lead-212, actinium-225, thorium-227, terbium-149 and terbium-161:

and wherein the at least one radionuclide is located in at least one of the pores, or a composition comprising said particle together with at least one pharmaceutically acceptable carrier, diluent, and/or excipient,

for use in a method for the treatment of a proliferative disease.

The particle is defined as disclosed herein, and may be a nanoparticle or a microparticle, preferably a nanoparticle.

The terms “treating” and “treatment” and “therapy” (and grammatical variations thereof) are used herein interchangeably, and refer to 1) inhibiting the disease; for example, inhibiting a disease, condition or disorder in a subject who is experiencing or displaying the pathology or symptomatology of the disease, condition or disorder, including prevention of disease (i.e. prophylactic treatment, arresting further development of the pathology and/or symptomatology), or 2) alleviating the symptoms of the disease, or 3) ameliorating the disease; for example, ameliorating a disease, condition or disorder in an subject who is experiencing or displaying the pathology or symptomatology of the disease, condition or disorder (i.e., reversing the pathology and/or symptomatology). The terms may relate to the use and/or administration of medicaments, active pharmaceutical ingredients (API), and/or pharmaceutical grade supplements.

As used herein, the terms “administer”, “administration”, and “administering” refer to (1) providing, giving, dosing and/or prescribing by either a health practitioner or their authorised agent or under their direction, or by self-administration, a formulation, preparation or composition according to the present disclosure, and (2) putting into, taking or consuming by the subject themselves, a formulation, preparation or composition according to the present disclosure.

As used herein, “subject” means any human or non-human animal selected for treatment or therapy, and encompasses, and may be limited to, “patient”. None of the terms should be construed as requiring the supervision (constant or otherwise) of a medical professional (e.g., physician, nurse, nurse practitioner, physician's assistant, orderly, clinical research associate, etc.) or a scientific researcher.

The subject is preferably a human subject. The subject may be male or female. In some embodiments, the subject is an adult (i.e. 18 years of age or older). In certain embodiments, the subject is geriatric. In certain embodiments, the subject is not geriatric. The subject is preferably a subject that has been diagnosed with a proliferative disease, such as a cancer.

The diseased tissue to be targeted may be at a soft tissue site, at a calcified tissue site or a plurality of sites which may all be in soft tissue, all in calcified tissue or may include at least one soft tissue site and/or at least one calcified tissue site. In one embodiment, at least one soft tissue site is targeted. The sites of targeting and the sites of origin of the disease may be the same, but alternatively may be different. Where more than one site is involved, this may include the site of origin or may be a plurality of secondary sites. The term “soft tissue” is used herein to indicate tissues which do not have a “hard” mineralised matrix. In particular, soft tissues as used herein may be any tissues that are not skeletal tissues. Correspondingly, “soft tissue disease” as used herein indicates a disease occurring in a “soft tissue” as used herein. The invention is particularly suitable for the treatment of cancers and “soft tissue disease” thus encompasses carcinomas, sarcomas, myelomas, lukemias, lymphomas and mixed type cancers occurring in any “soft” (i.e. non-mineralised) tissue, as well as other non-cancerous diseases of such tissue. Cancerous “soft tissue disease” includes solid tumours occurring in soft tissues as well as metastatic and micro-metastatic tumours. Indeed, the soft tissue disease may comprise a primary solid tumour of soft tissue and at least one metastatic tumour of soft tissue in the same patient. Alternatively, the “soft tissue disease” may consist of only a solid tumour or only metastases with the primary tumour being a skeletal disease.

When the particles or composition of the invention is used as a medicament and/or in a method of treatment according to the invention, the targeting moiety may be selected based on specific biomarkers, such as antigens, expressed by or near tissue, cells or organs affected by the proliferative disease.

In some embodiments, said proliferative disease is a cancer, a non-cancerous tumour, a neoplastic disease, or a hyperplastic disease.

In some preferred embodiments, said proliferative disease is a cancer. As used herein, the terms “cancer” and “tumour” refer to any neoplastic growth in a subject, including an initial tumour and any metastases. The cancer can be of the liquid or solid tumour type. Liquid tumours include tumours of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumours can originate in organs and include, but are not limited to, cancers of the lungs, brain, breasts, prostate, ovaries, colon, kidneys and liver.

In some embodiments, the cancer is selected from the list comprising or consisting of lung cancer, pancreatic cancer, colorectal cancer; liver cancer, glioma, renal cancer, non-hodgkin lymphoma, neuroblastoma, CNS metastases, peritoneal cancer, follicular lymphoma, colorectal cancer, small cell lung cancer, carcinoma, sarcoma, myeloma, leukemia, lymphoma, prostate cancer or mixed type cancer.

In specific embodiments, the cancer is a metastatic cancer. Treatment of metastatic cancers is notoriously difficult using conventional anticancer therapies, but the targeted MOF vehicles of the invention represent a promising line of treatment for such cancer.

The targeting moiety of the particles will be selected based on the particular disease to be treated. E.g.: CD37 is highly expressed on the majority of B-cells and B-cell lymphomas, is absent on normal stem cells and is lost again following differentiation into plasma cells. Because of its high prevalence on the surface of B-lymphomas, CD37 is a target for several different agents in clinical development. Thus, for such cancers, anti-CD37 may be a useful targeting moiety.

Non-Hodgkins lymphoma is difficult to treat with conventional therapy (surgery, radiation) as it is spreads around the lymph system. In addition, non-Hodgkin's lymphomas can metastase to other tissue types, which is another reason why the present invention can be especially effective against non-Hodgkin's lymphoma.

Further, the invention may also be useful against chronic inflammatory diseases such as rheumatoid arthritis, psoriatic arthritis, inflammatory bowel disease such as ulcerative colitis and/or Crohn's disease, and/or chronic obstructive pulmonary disease. Thus, in a further aspect, the invention relates to

A particle comprising
a MOF, wherein the MOF comprises
   a repeating three-dimensional network of inorganic monomers and organic monomers, forming pores, and
   at least one free functional group that extends into a pore, wherein the functional group is selected from the group comprising carboxylic acid, carboxylate, hydroxyl, sulfonic acid, sulfonate, sulfhydryl, primary amines, secondary amines, and combinations thereof;
optionally at least one targeting moiety connected to the particle on an external surface of the particle; and
at least one radionuclide, wherein the at least one radionuclide is selected from the group comprising radium-223, radium-224, radium-225, bismuth-212, bismuth-213, lead-212, actinium-225, thorium-227, terbium-149 and terbium-161:

and wherein the at least one radionuclide is located in at least one of the pores, or a composition comprising said particle together with at least one pharmaceutically acceptable carrier, diluent, and/or excipient,

for use in a method for the treatment of a chronic inflammatory disease.

In these embodiments, the subject is preferably a subject that has been diagnosed with a chronic inflammatory disease, such as rheumatoid arthritis, psoriatic arthritis, inflammatory bowel disease such as ulcerative colitis and/or Crohn's disease, and/or chronic obstructive pulmonary disease. The inflammatory process in the body serves an important function in the control and repair of injury. Commonly referred to as the inflammatory cascade, or simply inflammation, it can take two basic forms, acute and chronic. Acute inflammation, part of the immune response, is the body's immediate response to injury or assault due to physical trauma, infection, stress, or a combination of all three. Acute inflammation helps to prevent further injury and facilitates the healing and recovery process.

When inflammation becomes self-perpetuating, it can result in chronic or long-term inflammation. This is known as chronic inflammation, and lasts beyond the actual injury; sometimes for months or even years. It can become a problem itself, and require medical intervention to control or stop further inflammation-mediated damage. Unbalance of inflammatory and anti-inflammatory components of the immune system can result in chronic inflammatory disease. Rebalancing the immune system by killing the inflammatory cells with radioisotope loaded MOFs can prevent the chronic inflammation and treat the patients.

The particle or composition for use as a medicament and/or in a method of treatment according to the invention will be administered to a subject in a therapeutically effective dose. The term “therapeutically effective dose” as used herein means the amount of particle according to the invention which is effective for producing the desired therapeutic effect in a subject at a reasonable benefit/risk ratio applicable to any treatment. The therapeutically effective dosage amount may vary depending upon the route of administration and dosage form. Further, dosages may depend on the particle to be used, the type of radioactive decay of the radionuclide cation and/or its daughters, the stage of the condition, age and weight of the subject, etc. and may be routinely determined by the skilled practitioner according to principles well known in the art.

In some embodiments, the amount of radionuclide cation used per patient dosage may be in the range of from 1 kBq to 10 GBq, preferably 1 kBq to 100 MBq, more preferably 10 kBq to 25 MBq, even more preferably in the range of from 10 kBq to 10 MBq. The dosage and the maximum dosage may be determined by the person skilled in the art based on common general knowledge about suitable dosages and maximum dosages. It is accepted in the art that a realistic and conservative estimate of the toxic side effects of daughter isotopes must be adopted.

In some embodiments, the particles are administered at a dosage of 18 to 4000 kBq/kg bodyweight, such as at a dosage of 18 to 2000 kBq/kg bodyweight, e.g. at a dosage of 18 to 400 kBq/kg bodyweight, preferably 36 to 200 kBq/kg, such as 50 to 200 kBq/kg, more preferably 75 to 170 kBq/kg, especially 100 to 130 kBq/kg. In a study of the applicant, dosage ranges up to 1875 kBq/kg have been tested. Correspondingly, a single dosage unit may comprise around any of these ranges multiplied by a suitable bodyweight, such as 30 to 150 Kg, preferably 40 to 100 Kg (e.g. a range of 540 kBq to 4000 KBq per dose etc). The dosage, the particle and the administration route may be such that the dosage of progeny generated in vivo is less than 300 kBq/kg, such as less than 200 kBq/kg, preferably less than 150 kBq/kg, such as less than 100 kBq/kg.

The therapeutically effective dose of the particle or composition according to the invention can be administered in a single dose or in divided doses. The particle or composition according to the invention can be administered once, twice or more times a day, once every two days, once every three days, twice a week or once a week, or as deemed appropriate by a medical professional. In certain embodiments, the particle or composition according to the invention is administered once daily. In other embodiments, the particle or composition according to the invention is administered twice daily. In some embodiments, the dosage regimen is predetermined and the same for the entire patient group. In other embodiments, the dosage and the frequency of administration of treatment with the particle or composition according to the invention is determined by a medical professional, based on factors including, but not limited to, the stage of the disease, the severity of symptoms, the route of administration, the age, body weight, general health, gender and/or diet of the subject, and/or the response of the subject to the treatment.

In some embodiments, the therapeutically effective dose is administered at regular intervals. In other embodiments, the dose is administered when needed or sporadically. The particle or composition according to the invention may be administered by a medical professional. The particle or composition according to the invention may, depending on factors such as formulation and route of administration, be administered with food or without food. In some embodiments, the particle or composition according to the invention is administered at specific times of day.

The particle or composition for use as a medicament and/or in a method of treatment according to the invention may be administered locally or systemically. The particle or composition according to the invention may be administered by any administration route, including but not limited to, pulmonary, orally, intraperitoneally, intravenously, intramuscularly, intratumor, sublingually, subcutaneously, intrathecally, buccally, rectally, vaginally, occularly, nasally, transdermally, and cutaneously.

In some embodiments, the particle or composition is administered orally. In some embodiments, the particle or composition is administered with a meal or before a meal. In some embodiments, the particle or composition according to the invention is administered intravenously. In these embodiments, water is a particularly useful excipient. Saline solutions and aqueous dextrose and glycerol solutions can also be employed as liquid excipients, particularly for injectable solutions.

Preferred unit dosage formulations are those containing a therapeutically effective dose, as hereinbefore recited, or an appropriate fraction thereof, of a particle according to the invention. A composition of the invention may be presented in unit dosage form as a single dose wherein all active and inactive ingredients are combined in a suitable system and components do not need to be mixed before administration. Alternatively, a composition may be presented as a kit such as disclosed above, and may contain instructions for storing, preparing, administering and/or using the composition.

In some embodiments, the duration of the use of the particle or composition for use as a medicament and/or in a method of treatment according to the invention is determined by the observed effect of the treatment, such as by reduction of and/or amount of target antigen expression. In some embodiments, treatment is sustained until no further improvement can be expected. In certain embodiments, the duration of the treatment with the particle or composition according to the invention is at least two weeks, at least one month, at least three months, such as three months, six months, nine months, a year, three years, five years. In other embodiments, the duration is determined by a medical professional, based on factors including but not limited to the nature and severity of the symptoms, the route of administration, the age, body weight, general health, gender and/or diet of the subject, and/or the response of the subject to the treatment.

In certain embodiments, the particle or composition according to the invention is administered alone. In other embodiments, the particle or composition according to the invention is administered in combination with one or more other therapeutic agents. Said one or more other therapeutic agents may be known to have an effect against a proliferative disease, such as cancer, and/or may have an additive or synergistic mechanism of action on treatment of said proliferative disease, such as a cancer, together with the particle or composition of the invention. In some embodiments, the particle or composition according to the invention is administered as part of a combination therapy. Combination therapies comprising a particle or composition according to the invention may refer to compositions that comprise the particle or composition according to the invention in combination with one or more therapeutic agents, and/or co-administration of the particle or composition according to the invention with one or more therapeutic agents wherein the particle or composition according to the invention and the other therapeutic agent or agents have not been formulated in the same composition. When using separate formulations, the particle or composition according to the invention may be administered simultaneously, intermittent, staggered, prior to, subsequent to, or combinations of these, with the administration of another therapeutic agent.

The embodiments and features described in the context of one aspect, e.g. for the aspect directed to the particle or the composition, also apply to the other aspects of the invention, such as the use thereof as a medicament or in a method of treatment.

In a further aspect, the invention provides a method of treatment, the method comprising the step of administering an effective amount of a particle or composition of the invention, to a subject in need thereof.

In a further aspect, the invention provides a method of treatment of a proliferative disease, the method comprising the step of administering an effective amount of a particle or composition of the invention, to a subject in need thereof.

In a further aspect, the invention provides a method of treatment of chronic inflammatory disease, the method comprising the step of administering an effective amount of a particle or composition of the invention, to a subject in need thereof.

In a further aspect, the invention provides the use of a particle or composition of the invention as a medicament.

In a further aspect, the invention provides the use of a particle or composition of the invention for treatment of a proliferative disease.

In yet a further aspect, the invention provides the use of a particle or composition of the invention for treatment of a of chronic inflammatory disease.

The invention shall not be limited to the shown embodiments and examples. While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is to be understood that various alternatives to the embodiments described herein can be employed in practicing the disclosure.

It is to be understood that every embodiment of the disclosure can optionally be combined with any one or more of the other embodiments described herein.

It is to be understood that each component, compound, particle, or parameter disclosed herein is to be interpreted as being disclosed for use alone or in combination with one or more of each and every other component, compound, or parameter disclosed herein. It is further to be understood that each amount/value or range of amounts/values for each component, compound, or parameter disclosed herein is to be interpreted as also being disclosed in combination with each amount/value or range of amounts/values disclosed for any other component(s), compound(s), or parameter(s) disclosed herein, and that any combination of amounts/values or ranges of amounts/values for two or more component(s), compound(s), or parameter(s) disclosed herein are thus also disclosed in combination with each other for the purposes of this description. Any and all features described herein, and combinations of such features, are included within the scope of the present invention provided that the features are not mutually inconsistent.

It is to be understood that each lower limit of each range disclosed herein is to be interpreted as disclosed in combination with each upper limit of each range disclosed herein for the same component, compound, or parameter. Thus, a disclosure of two ranges is to be interpreted as a disclosure of four ranges derived by combining each lower limit of each range with each upper limit of each range. A disclosure of three ranges is to be interpreted as a disclosure of nine ranges derived by combining each lower limit of each range with each upper limit of each range, etc. Furthermore, specific amounts/values of a component, compound, or parameter disclosed in the description or an example is to be interpreted as a disclosure of either a lower or an upper limit of a range and thus can be combined with any other lower or upper limit or a range or specific amount/value for the same component, compound, or parameter disclosed elsewhere in the application to form a range for that component, compound, or parameter.

### EXAMPLES

### Example 1: Conjugation of Anti-CD37 IgG to UIO-66-(COOH)₂

The UIO-66-(COOH)₂ MOF was prepared by a method based on the method of Zhiijie Chen et al, CrystEngComm 2019, 14, 2409-2415, however an oxychloride salt was used rather than an oxynitrate salt as a precursor for the MOF. UIO-66-(COOH)₂ antibody conjugation was assessed by analysing UIO-66-(COOH)₂ antibody conjugated particles by bicinchoninic acid assay (BCA). The BOA Protein Assay combines the well-known reduction of Cu²⁺ to Cu¹⁺ by protein in an alkaline medium with the highly sensitive and selective colorimetric detection of the cuprous cation (Cu¹⁺) by bicinchoninic acid (BOA). The first step is the chelation of copper with protein in an alkaline environment to form a light blue complex. In this reaction, known as the biuret reaction, peptides containing three or more amino acid residues form a coloured chelate complex with cupric ions in an alkaline environment containing sodium potassium tartrate.

In the second step of the colour development reaction, BOA reacts with the reduced (cuprous) cation that was formed in step one. The intense purple-coloured reaction product results from the chelation of two molecules of BOA with one cuprous ion.

Procedure:

5 mg of UiO-66-(COOH)₂ was dissolved in 0.05 M 2-(N-morpholino)ethanesulfonic acid (MES) buffer, pH 6.3, treated with 1.2 mg 1-ethyl-3-(3-dimethylaminopropyl)carbodiimide (EDC) and 1.2 mg N-hydroxysulfosuccinimide(sulfo-NHS) for 15 min, before the particles were washed twice times with 0.05 M MES buffer (pH 6.3). The particles were then resuspended in 0.05 M MES buffer (pH 6.3), containing 100 μg of anti-CD37 IgG. The reaction mixture was incubated with continuous rocking for 3 hours. The MOF-antibody particles were washed with 0.05 M MES buffer (pH 6.3) and resuspended in 100 mM Tris buffer prior to washing and storing the particles with 0.05 M MES buffer (pH 6.3).

Antibody (IgG) conjugated UIO-66(COOH)₂ and an albumin standard was assessed by the BCA assay, shown in FIG. 12. The trendline equation from the albumin was used to calculate the protein concentration of the UIO-66(COOH)₂-IgG particles per mL: Absorbance—blank:0.096, corresponding to 77 μg/mL (77% of the antibody input).

### Example 2: Structural Stability and Barium Retention Ability of a MOF in Human Serum

In this experiment, barium(II) was used as a mimic for radium(II). Ba(II) and Ra(II) have similar ionic radius (162(8) pm for Ra^{2+,} 149 pm for Ba²⁺) and orbital structure (both members of the alkaline-earth metal group and have noble gas electron configuration in neighbouring periods), and are expected to behave similarly in chemical and biological systems.

Procedure:

200 mg UiO-66-(COOH)₂ was washed once with 0.05 M MES buffer (pH 6.3) and suspended in 40 mL aqueous solution of barium(II)acetate (14.7 ug Ba/mL). The suspension was agitated at room temperature for 60 minutes. The MOF was recovered, washed once with 5 mL 0.05 mol/L MES buffer (pH 6.3) before being suspended in human serum. The MOF/serum suspension was kept stirring at room temperature for 11 days. Samples were taken after 15 minutes, plus after 1, 2, 4, and 7 days. Elemental analysis was carried out using the MP-AES method following digestion of serum samples using a 1:1 volumetric mixture of HNO3 (65%) aq. And H₂O₂ (33%) aq at 70C for 1 hour.

Elemental analysis showed that the MOF adsorbed 97.2% of the Ba²⁺ in solution, giving a Ba²⁺ concentration in the MOF at 2.87 ug/mg. (0.287% wt.). Analysis of serum samples taken after 15 minutes, plus after 1, 2, 4, and 7 days, showed a total leakage of Ba²⁺ into the serum of 6.6%, 8.4%, 9.4%, 8.3% and 5.5%, respectively. This indicates that, after initial leakage, the MOF contains all the Ba²⁺.

The MOF was separated from the serum after 11 days and analysed by powder X-ray diffraction and energy dispersive X-ray spectroscopy. Powder X-ray diffraction patterns of UiO-66-(COOH)₂ as received and after a 11-day stability test clearly show that the MOF crystal structure is still intact. EDS shows that the mass ratio of Ba/Zr in the MOF is 1.10%, closely matching the value measured by MP-AES (0.287% wt.) compared to the theoretical mass percentage of Zr in the MOF (25.1%, from the formula Zr6O4(OH)4(COO)12(C8H4O4)6), 1.14%, within the margin of error of the experiments.

| ID | ppm |
|---|---|
| [Ba2+] in MOF-serum suspension | 19.10889 |
| [Ba2+] in serum after 15 min | 1.259 |
| [Ba2+] in serum after 24 h | 1.608 |
| [Ba2+] in serum after 48 h | 1.802 |
| [Ba2+] in serum after 96 h | 1.5955 |
| [Ba2+] in serum after 168 h | 1.0475 |

Table 1 shows the barium concentrations found in experiment 2.

Hence, the experiment shows that the MOF particle in itself is stable in human serum for at least 11 days, and that the chelation of barium cations in the MOF particle is stable for at least 11 days. Incidentally, the experiments also show the MOF's ability to adsorb substantial quantities of Ca2+. K+ and Na+ without leaking Ba2+.

### Example 3: Structural Stability and Barium Retention Ability of a MOF in Human Serum

This experiment corroborates the results from Experiment 2, using a different Ba2+ source and two different concentrations.

Procedure:

2 separate portions of 120 mg UiO-66-(COOH)₂ were washed three times with 5 mL 0.05 mol/L MES buffer (pH 6.3) and then suspended in two 5 mL separate aqueous solutions of barium(II)nitrate (1.165 mg/mL Ba, 0.109 mg/mL Ba, respectively). The suspensions were agitated at room temperature for 90 minutes. The MOF was recovered, washed once with 5 mL 0.05 mol/L MES buffer (pH 6.3) before being suspended in human serum. Samples were taken after 24 and 48 hours. The MOF/serum suspension was kept stirring at room temperature for 2 days. Samples were taken after 24 and 48 hours. Elemental analysis was carried out using the MP-AES method following digestion of serum samples using a 1:1 volumetric mixture of HNO3 (65%) aq. And H₂O₂ (33%) aq at 70C for 1 hour.

From the 1.165 mg/mL Ba solution: Elemental analysis showed that the MOF adsorbed 98.8% of the Ba2+ in solution, giving a Ba2+ concentration in the MOF at 48.0 ug/mg. (4.8% wt.). Analysis of serum samples taken after 24 and 48 hours, showed a total leakage of Ba2+ into the serum of 8.8% and 9.2%, respectively. This indicates that there was negligible leaking of Ba2+ between 24 and 48 hours. EDS shows that the mass ratio of Ba/Zr in the MOF is 19.4%, closely matching the value measured by MP-AES (4.8 wt.) compared to the theoretical mass percentage of Zr in the MOF (25.1%, from the formula Zr6O4(OH)4(COO)12(C8H4O4)6), 19.1%, within the margin of error of the experiments.

From the 0.109 mg/mL Ba solution: Elemental analysis showed that the MOF adsorbed 92.0% of the Ba2+ in solution, giving a Ba2+ concentration in the MOF at 4.19 ug/mg. (0.42% wt.). Analysis of serum samples taken after 24 and 48 hours, showed a total leakage of Ba2+ into the serum of 6.4% and 6.0%, respectively. This indicates that there was no leaking of Ba2+ between 24 and 48 hours. EDS shows that the mass ratio of Ba/Zr in the MOF is 1.43%, closely matching the value measured by MP-AES (0.42 wt.) compared to the theoretical mass percentage of Zr in the MOF (25.1%, from the formula Zr6O4(OH)4(COO)12(C8H4O4)6), 1.67%, within the margin of error of the experiments.

| | Solution 1 | Solution 2 |
|---|---|---|
| ID | (ppm) | (ppm) |
| [Ba2+] in MOF-serum suspension | 575.505 | 50.23833 |
| [Ba2+] in serum after 24 h | 50.4425 | 3.213 |
| [Ba2+] in serum after 48 h | 52.7845 | 3.027 |

Table 2 shows the barium concentrations found in experiment 3.

### Example 4: MOF-IgG Cell Interaction

This experiment shows that anti-CD37-conjugated MOFs (NP1) bind CD37 positive cells (Duadi cells). The Daudi cell line is composed of B lymphoblasts isolated from the peripheral blood of a Burkitt's Lymphoma patient, illustrating that the anti-CD37-conjugated MOFs can identify blood derived cancer cells.

Procedure:

66 000 Daudi cells were transferred to flow tubes containing: phosphate buffered saline (PBS), isotype control, UiO-66-(COOH)₂ anti-CD37 stability study sample (incubated at 20 degree C. for 6 months) or UiO-66-(COOH)₂ anti-CD37 freshly conjugated. Samples were incubated with continuous mixing for 30 minutes prior to adding 400 μL of 10% fetal bovin serum (FBS) in PBS and centrifugating at 350 g for 8 min. The supernatant was discarded prior to adding 100 μL anti-CD37-PE (10 μL anti-CD37-PE mixed with 90 μL PBS) to the PBS sample, or 100 μL anti-mouse-PE (10 μL anti-mouse-PE mixed with 90 μL PBS) to the isotype control and UiO-66-(COOH)₂ aCD37 samples. Samples were incubated with continuous mixing for 30 minutes prior to adding 400 μL of 10% FBS in PBS and centrifugating at 350 g for 8 min. The supernatant was discarded prior to adding 400 μL 10% FBS in PBS and flow cytometry analysis.

The stability control sample used in this experiment was prepared by storing UiO-66-(COOH)₂ anti-CD37 at 20 degrees for 6 months.

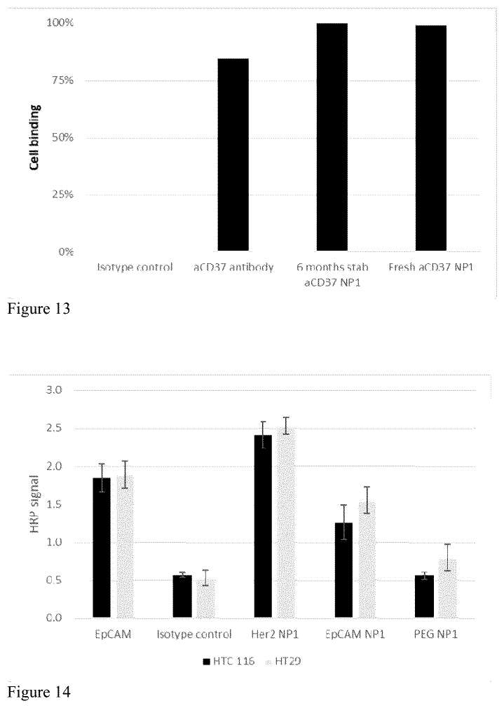

FIG. 13 provides the results showing that anti-CD37-conjugated MOFs (NP1) binds CD37 positive cells and is stable for at least 6 months at room temperature.

### Example 5: MOF-Anti-EpCAM and MOF-Anti-HER2 Cell Binding to Colorectal Cell Lines

This experiment shows that anti-EpCAM or anti-HER-conjugated UiO-66-(COOH)₂ bind colorectal cancer cells (HTC116 and HT29) expressing both antigens. HTC116 cells originates from the colon of an adult male, colon cancer patient. And HT29 cell line is derived from the primary tumour of a female patient with colorectal adenocarcinoma. Thus, illustrating that both anti-EpCAM and anti-HER-antibodies (IgG) conjugated UiO-66-(COOH)₂ can identify colorectal derived cancer cells.

Procedure:

Cells were seeded at 50 000 cells per well in 96-well plates and cultured overnight in complete 10% Fetal bovine serum (FBS) Roswell Park Memorial Institute (RPMI) cell culture media. The next day, media was discarded from each well prior to adding 2 μg anti-EpCAM, 2 μg isotype control or 15 μL UiO-66-(COOH)₂ (NP1) diluted in prewarmed cell culture media to the total volume of 200 μL per well. The samples were incubated for 30 min at 37 degree C. prior by discarding the media and adding 200 μL PBS (phosphate buffered saline) twice gently. 100 μL of PBS and 100 μL anti-human IgG-HRP solution (2.5 μL anti-human IgG-HRP solution/mL) was added per sample and incubated for 30 min at room temperature. After secondary staining, cells were washed with PBS twice gently prior to adding 100 μL of TMB (3,3′,5,5′-Tetramethylbenzidine) HRP (horseradish peroxidase) substrate solution to each well and incubating for 15 min. 100 μL of stopping solution was added per well to stop the reaction prior to measuring the absorbance of each well at 450 nm. The absorbance is reported in the graph as “HRP signal”. FIG. 14 shows that anti-EpCAM and anti-HER2 UiO-66-(COOH)₂ bind colorectal cancer cells (HTC116 and HT29).

### Example 6: MOF-Anti-HER2 Breast Cancer Cell Interaction

This experiment shows that anti-HER2-conjugated UiO-66-(COOH)₂ binds HER2 positive cancer cells (JIMT1 cells). JIMT1 cells are epithelial cells originating from a female patient with breast ductal adenocarcinoma. Thus, JIMT1 cell interaction illustrates how anti-HER2-conjugated UiO-66-(COOH)₂ can identify breast derived cancer cells.

Procedure: The same procedure was used for JIMT1 cell interaction as for colorectal cancer cell interaction, see example 5 (FIG. 14).

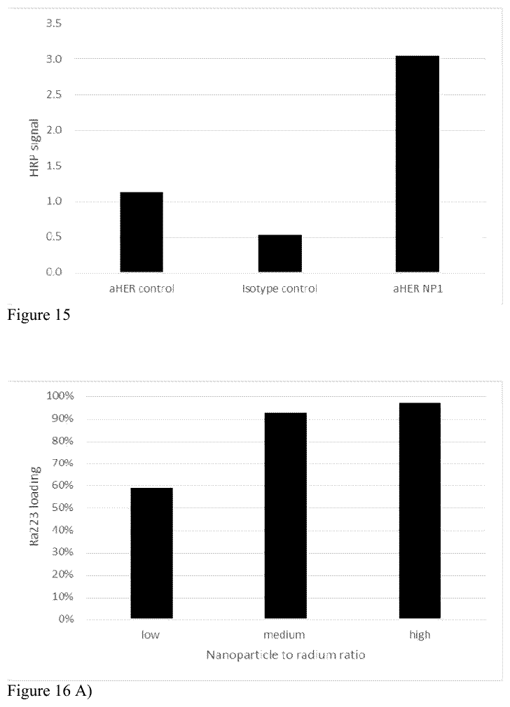

FIG. 15 shows the anti-HER2-conjugated UiO-66-(COOH)₂ binds to breast cancer cells, JIMT1 cells.

### Example 7: Radium 223 (Ra223) Adsorption and Retention Ability of a MOF in Human Serum

In this experiment, UiO-66-(COOH)₂ was loaded with Ra223 to demonstrate adsorption and retention in a relevant environment (serum). UiO-66-(COOH)₂ adsorbed Ra223, however, as Xofigo was used as the Ra223 source, presence of Na+ most likely affected the adsorption efficiency. Increasing UiO-66-(COOH)₂ concentration improved adsorption efficiency (from 59% to 97%), and it is highly likely that using pure Ra223 would also improve adsorption when using low UiO-66-(COOH)₂ concentrations. The serum challenge of Ra223 loaded UiO-66-(COOH)₂ demonstrated retention capacity of 94% during a five-day period.

Procedure:

0.0085 mg (low), 0.085 mg (medium) and 0.85 mg (high) UiO-66-(COOH)₂ conjugated to anti-HER2 antibody and polyethylene glycol (PEG) was mixed with 10 kBq Ra223 and agitated at room temperature for 60 minutes. The MOF was recovered, washed twice with distilled water before measuring the total gamma emitting activity with an Hidex instrument (A). After discarding the supernatant, the remaining pellet of the 0.85 mg (high) UiO-66-(COOH)₂ was suspended in fetal bovine serum (FBS). The MOF/serum suspension was kept at room temperature for 5 days analysing the total supernatant after centrifugation at day 1, replenishing the pellet in FBS and sampling the supernatant at day 5 after centrifugation (B).

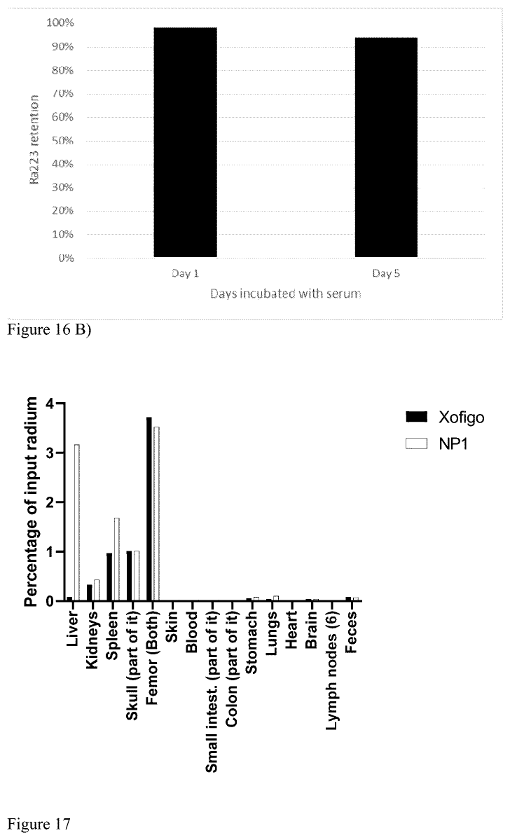

Results: Hidex analysis showed that UiO-66-(COOH)₂ adsorbed 59-97% of the Ra223 in solution, ref. FIG. 16A). And further that the highest dose of UiO-66-(COOH)₂ retained 94% of Ra 223 after 5 days serum challenge, ref. FIG. 16 B).

### Example 8: Biodistribution of Radium 223 (Ra223) Loaded UiO-66-(COOH)₂ in Healthy Mice

This experiment shows how Ra223 is contained by the MOF prototypes in vivo, demonstrating a different biodistribution compared to Ra223 (Xofigo). As described under example 7, the presence of Na+ in Xofigo most likely drastically affects the in vivo retention of radium loaded UiO-66-(COOH)₂ (NP1) resulting in radium traveling to the bone.

Procedure:

10 kBq Ra223/0.085 mg MOF was incubated for 24 hours prior to washing (centrifugation and discarding supernatant prior to adding distilled water) and resuspended in physiological salt water for injection into healthy mice (475 kBq per kg mice). In a control mouse 475 kBq per kg of Xofigo was injected at the same time. The mice were sacrificed 18 hours post injection before harvesting, liver, kidneys, spleen, skull, femur, skin, blood, small intestine, large intestine, stomach, lungs, heart, brain and lymph nodes.

Most major organs (as designated in FIG. 17) were analysed by gamma emission analysis with HIDEX, ref. FIG. 17. Ra223 loaded nanoparticles protect Ra223 from accumulation into the bone tissue.

### Example 9. Safety Tolerance of UiO-66-(COOH)₂ Loaded with Ra223 In Vivo

Maximum tolerable dose (MTD) experiments were performed to determine maximum tolerable dose, which was and will be used for biodistribution and treatment experiments. Weight loss, and red spots was observed for some of the mice injected with 937 kBq or 1875 kBq per kg and was sacrificed. Weight loss is an objective measurement of side effects, and no weight loss implies that the mice are healthy. Thus, under these conditions the MTD was 475 kBq per kg.

Procedure:

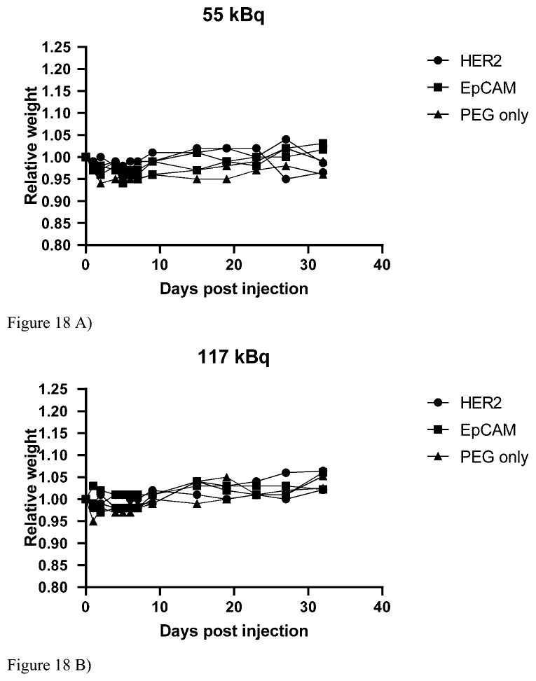

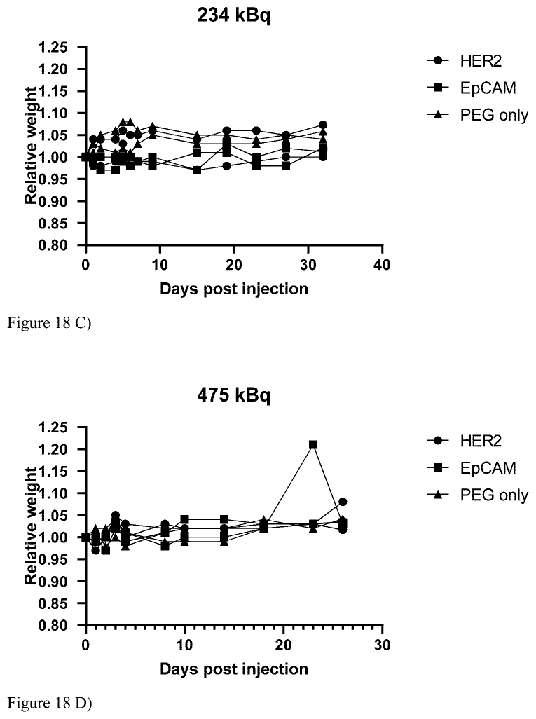

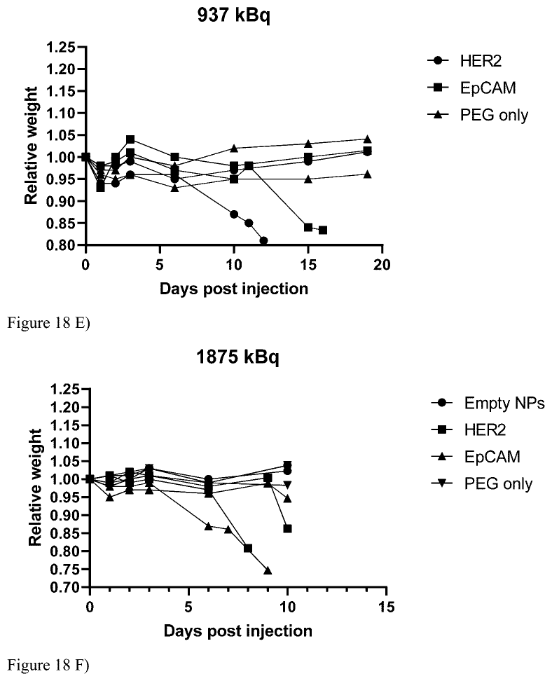

We here defined MTD as the maximum tolerable dose where the mice still live without too severe side effects. 6 dose levels were used, starting with 55 kBq/kg, and doubling the dose until 1870 kBq/kg with 2 mice at each dose level per prototype (UiO-66-(COOH)₂ anti-HER₂, UiO-66-(COOH)₂ anti-EpCAM or UiO-66-(COOH)₂ PEG). Ref FIG. 18. shows that mice injected with 937 or 1875 kBq radium loaded nanoparticles did lose significant amount of weight and was sacrificed. Thus, MTD was set to 475 kBq which is lower compared to what has been observed with pure radium in other studies. This implies (as observed in biodistribution experiments) that more radium is circulated in the body relative to pure radium which is secreted more frequently. Injection of a high dose of empty UiO-66-(COOH)₂ PEG (10 mg/kg) did not results in symptoms or weight loss.

### Example 10. Viability Test of UiO-66-(COOH)₂ (NPs) Conjugated with PEG Demonstrate No Toxicity In Vitro

Exposing HT29 and HCT116 with an increasing dose of NPs over two days before analysing the cell viability demonstrate that NP does not induce cell death under these conditions.

Procedure:

HT29 or HCT116 was exposed to 0.001 to 500 μg per well of UiO-66-(COOH)₂ (NPs) and cultured at 37 degree C. at 5% CO₂ in a humidified incubator for 24 and 48 hours prior to discarding the supernatant and adding 100 μL fresh cell culture media (10% Fetal bovine serum (FBS) Roswell Park Memorial Institute (RPMI)) and 20 μL the CellTiter 960 AQueous One Solution Reagent (Promega) containing tetrazolium compound [344,5-dimethylthiazol-2-yl)-5-(3-carboxymethoxyphenyl)-2-(4-sulfophenyl)-2H-tetrazolium, inner salt; MTS] and an electron coupling reagent (phenazine ethosulfate; PES). Each plate was incubated at 37 degree C. at 5% CO₂ in a humidified incubator for 1.5 hours before measuring the absorbance at 490 nm in a plate reader.

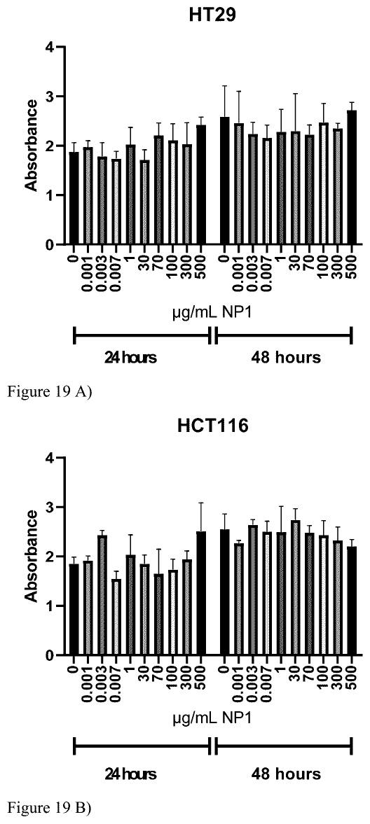

Ref FIGS. 19A and B) the absorbance was unchanged under all conditions thus implying that the cell content per well was unchanged over the period of 48 hours.

### Example 11. Planned Experiment: Biodistribution of UiO-66-(COOH)₂ (NPs) Conjugated to Anti-HER₂, Anti-EpCAM Antibodies and Polyethylene Glycol (PEG) to Investigate the Tumour Targeting of NPs with and without Antibodies

Procedure:

The biodistribution of UiO-66-(COOH)₂ targeting EpCAM or HER2 will be investigated, along with their ability to localize to the tumours using tumour models representing HER2+ breast cancer and EpCAM colorectal cancer. To investigate the specificity of the nanoparticles (NPs), the NPs with and without antibodies attached to the particles will be used in addition to radioactivity. The mice will be sacrificed from day 0 to 30 post treatment to investigate the drug's biodistribution over time, and how specific the biodistribution will be considering the tumour. Mice will be sacrificed at each timepoint with each drug. For the two EpCAM targeting drugs this will be investigated in two or more models (for example HCT116 and HT29), with tumour growing in for example the liver and intraperitoneally. For tumour growing intraperitoneally, the substances will be injected either intra Intravenous (iv) or Intraperitoneal (ip) to investigate which injection form has the best biodistribution with regards to tumour growing in the peritoneal cavity. The two HER2 targeting drugs will be investigated in at least two models, for example SKBR3 (cell line) and BBRC160 (PDX) resulting in breast cancer, liver tumours and peritoneal tumours.

### Example 12. Planned Experiment: Effect Studies of UiO-66-(COOH)₂ (NPs) Conjugated to Anti-HER2, Anti-EpCAM Antibodies and Polyethylene Glycol (PEG) to Investigate the Tumour Killing of NPs with and without Antibodies

Procedure:

The ability of the UiO-66-(COOH)₂ to reduce tumour growth will be tested in two tumour models representing HER2+ breast cancer (SKBR3 and BB6RC160) and three EpCAM+ colorectal tumours (HCT116, HT29 and SW620). As colorectal cancer frequently metastasizes to the liver and peritoneal cavity, it is relevant to investigate the efficacy of UiO-66-(COOH)₂ in these settings, as they have a more similar microenvironment to tumour in patients.

1. A particle comprising
a MOF, wherein the MOF comprises
a repeating three-dimensional network of inorganic monomers and organic monomers, forming pores, and
at least one free functional group that extends into a pore, wherein the functional group is selected from the group consisting of carboxylic acid, carboxylate, hydroxyl, sulfonic acid, sulfonate, sulfhydryl, primary amines, secondary amines, and combinations thereof;
optionally at least one targeting moiety connected to the particle on an external surface of the particle; and
at least one radionuclide, wherein the at least one radionuclide is selected from the group consisting of radium-223, radium-224, radium-225, bismuth-212, bismuth-213, lead-212, actinium-225, thorium-227, terbium-149 and terbium-161:
and wherein the at least one radionuclide is located in at least one of the pores.

2. The particle according to claim 1, wherein the MOF is UiO-66-COOH or UiO-66-(COOH)₂.

3. The particle according to claim 1, wherein the radionuclide is a radionuclide cation selected from the group consisting of radium-223, radium-224, radium-225, bismuth-212, bismuth-213, lead-212, thorium-227, terbium-149 and terbium-161.

4. The particle according to claim 1, wherein the at least one radionuclide is radium-223.

5. The particle according to claim 1, wherein the particle is a nanoparticle.

6. The particle according to claim 1, wherein at least one targeting moiety is present.

7. The particle according to claim 1, wherein at least one targeting moiety is present and is an antibody.

8. A kit comprising,
in a first container,
a particle comprising
a MOF, wherein the MOF comprises
a repeating three-dimensional network of inorganic monomers and organic monomers, forming pores, and
at least one free functional group that extends into a pore, wherein the functional group is selected from the group consisting of carboxylic acid, carboxylate, hydroxyl, sulfonic acid, sulfonate, sulfhydryl, primary amines, secondary amines, and combinations thereof;
optionally at least one targeting moiety connected to the particle on an external surface of the particle; and
and in a second container,
a radionuclide, wherein the radionuclide is selected from the group consisting of radium-223, radium-224, radium-225, bismuth-212, bismuth-213, lead-212, actinium-225, thorium-227, terbium-149 and terbium-161, or a radionuclide generating any one of radium-223, radium-224, radium-225, bismuth-212, bismuth-213, lead-212, actinium-225, thorium-227, terbium-149 and terbium-161 as a daughter.

9. A composition comprising at least one particle according to claim 1 together with at least one pharmaceutically acceptable carrier, diluent, and/or excipient.

10. The particle according to claim 1 for use as a medicament.

11. A method for the treatment of a proliferative disease, comprising administering a therapeutically effective amount of the particle according to claim 1 to a subject in need thereof.

12. The method according to claim 11, wherein the proliferative disease is cancer.

13. The method according to claim 12, wherein the cancer is a carcinoma, sarcoma, myeloma, leukaemia, lymphoma or mixed type cancer.

14. The method according to claim 11, wherein the proliferative disease is a hyperplastic or neoplastic disease.

15. A method for the treatment of a chronic inflammatory disease, comprising administering a therapeutically effective amount of the particle according to claim 1 to a subject in need thereof.

16. A method for the treatment of a liver cancer, comprising administering a therapeutically effective amount of the particle according to claim 1 to a subject in need thereof, wherein the particle does not comprise any targeting moiety.
